(19) United States
(12) Patent Application Publication
(10) Pub. No.: US 2003/0139700 A1
(43) Pub. Date: Jul. 24, 2003

(54) User interface for an automated radioisotope system

(75) Inventors: Daniel M. Elliott, Shorewood, MN (US); George M. Hoedeman, Edina, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Mentor Corporation

(21) Appl. No.: 10/010,734

(22) Filed: Nov. 13, 2001

Related U.S. Application Data
(60) Provisional application No. 60/247,482, filed on Nov. 10, 2000.
(60) Provisional application No. 60/247,229, filed on Nov. 10, 2000.

(51) Int. Cl. A61M 31/00

(52) U.S. Cl. 604/62

(57) ABSTRACT

An automated system for ejecting low dose radioisotope seeds into an implant needle includes a station connected to at least one source of radioisotope seeds. The station includes a computer processor that has a user interface that displays information about the automated system and accepts commands from a user to control the process of ejecting the radioisotope seeds into the implant needles. The user interface allows a user to alter a predetermined dose plan during the process of loading the implant needles. Preferably, the user interface is a touch-screen interface that displays a graphic representation of the coordinates of a plurality of locations, with the user selecting the next location by touching one of the coordinates. As the coordinate is touched, the icon associate with that coordinate would change color indicating that that location has been processed. The station may be either a loading station for loading a plurality of implant needles that are to be used in a manual implant procedure, or an automated station for implanting radioisotope seeds as part of an automated implant procedure.

### RELATED APPLICATIONS

The present application claims priority to two provisional applications filed Nov. 10, 2000, the first of which is entitled “USER INTERFACE FOR AN AUTOMATED RADIOISOTOPE SYSTEM”, Application No. 60/247,482, and the second of which is entitled “AUTOMATED IMPLANTATION SYSTEM FOR RADIOISOTOPE SEEDS”, Application No. 60/247,229. The present invention is a continuation-in-part of two co-pending applications that are commonly assigned to the assignee of the present invention, the first of which is entitled “AUTOMATED RADIOISOTOPE SEED LOADER SYSTEM FOR IMPLANT NEEDLES,” application Ser. No. 09/587,624, filed Jun. 5, 2000, and the second of which is entitled “AUTOMATED RADIOISOTOPE SEED CARTRIDGE,” application Ser. No. 09/587,642, filed Jun. 5, 2000.

### FIELD OF THE INVENTION

The present invention relates generally to the field of medical devices for handling radioisotope materials. More specifically, the present invention relates to a user interface for an automated system for handling low dose radioisotope seeds as part of a brachytherapy procedure or the like.

### BACKGROUND OF THE INVENTION

The use of radioisotopes for various medical procedures such as brachytherapy and the like is well known. Such uses fall into two general categories: (i) high dose radioisotopes which are temporarily positioned in relation to a patient's body for a relatively short period of time to effect the radiation treatment, and (ii) low dose radioisotopes which are permanently implanted in a patient's body with the duration of the radiation treatment determined by the strength and half-life of the radioisotope being implanted. High dose radioisotopes are typically implanted using a catheter arrangement and a device commonly known as an afterloader that advances the high dose radioisotope located on the end of a source wire through the catheter to the desired location. Low dose radioisotopes, on the other hand, are implanted using an array of implant needles with the low dose radioisotopes being encapsulated in very small containers known as seeds that are manually loaded into a series of implant needles and then ejected to form a three-dimensional grid of radioisotopes in the patient that corresponds to a dose plan as determined by the physician. The goal of the low dose brachytherapy procedure is to position this three-dimensional grid of radioisotopes seeds in and around a target cancerous tissue area. Each of the radioisotope seeds consists of a radioactive source such as Iodine (I-125) or Palladium (Pd-103) inside a small tube-like titanium shell that is about the size of a grain of rice. These type of low dose radioactive sources emit a very low energy radiation that is primarily absorbed by the tissue immediately surrounding the radioisotope seed. This constant low energy radiation is typically emitted by the radioisotope seeds for a period of up to six months as a way to kill the cancer cells in the target area without having to subject the patient to the discomfort and risks that often accompany high dose radioisotope procedures.

One common brachytherapy procedure is the use of low dose radioisotopes to treat prostate cancer. Although brachytherapy procedures using low dose radioisotopes can be applied to many different parts of the body, it is helpful to describe a particular treatment to gain a better understanding of these treatments. In a typical prostate cancer procedure, a predetermined number of seeds (between 1-6) are positioned within each of a series of implant needles (up to 40), with the seeds being spaced apart in each needle by small spacers. A small amount of bone wax is positioned on the tip of the implant needles to prevent the seeds and spacers from falling out until they are implanted in the patient. The loaded implant needles are then positioned at the appropriate location for insertion into the perineal area of the patient using a stand that has an X-Y coordinate grid. Each needle is manually positioned in the appropriate chamber in the grid and is inserted into the patient. An ultrasound probe is used to assist the physician in guiding each of the needles to the desired location. The seeds and spacers are delivered from the tip of the implant needle using a stylet and hollow needle arrangement where the hollow needle is preferably retracted while the stylet remains in place. When completed, the implanted seeds form a three-dimensional grid of radioisotope sources that implements a predetermined dose plan for treating the prostate cancer in the patient. For a more detailed background of the procedures and equipment used in this type of prostate cancer treatment, reference is made to U.S. Pat. No. 4,167,179.

Over the years there have been numerous advancements in the design of equipment for use in radioisotope procedures. U.S. Pat. Nos. 4,086,914, 5,242,373, 5,860,909, 6,007,474, 6,102,844, and 6,213,932, describe manual seed injector arrangements for a low dose radioisotope procedure that utilize drop-in seed cartridges or seed magazines to supply the seeds directly to an implant needle that is specifically adapted to such cartridges or magazines. U.S. Pat. No. 6,221,003 describes an elongated cartridge with a central channel that contains a plurality of seeds interspersed with a plurality of spacers for loading a single implant needle; however, the seeds and spacers are manually loaded into the central channel using leaded gloves or tweezers. PCT Publ. No. WO 01/66185 describes an alternative arrangement for loading a single implant needle in which a separate seed cartridge and spacer cartridge are manually advanced into corresponding slots in a loading tube such that a manually-operated plunger can dislodge the seed and spacer from chambers in the cartridges to load the implant needle. U.S. Pat. Nos. 4,150,298, 5,147,282, 5,851,172 and 6,048,300 describe replaceable cartridge assemblies that contain the source wire used in conjunction with specifically adapted afterloaders that advance the source wire into a catheter system for high dose radioisotope procedures.

Although such replaceable cartridges have been well received for use in connection with high dose radioisotope procedures, the standard techniques for low dose radioisotope procedures continue to utilize a series of preloaded implant needles that are manually loaded by a radiophysicist at the hospital just prior to the procedure. There are several reasons for why manual loading of the implant needles just prior to use in low dose radioisotope procedures is preferred. First, there are differences in the types of radioisotope sources that do not favor use of a cartridge arrangement for low dose radioisotope procedures. The source wires used for high dose radioisotope procedures use only one or a small number of very high power radioisotope sources having relatively long half-lives. As a result, it is cost effective and practical to provide for a cartridge arrangement for such a small number of high dose radioisotopes that can be preordered and maintained at the hospital well in advance of a procedure. In contrast, given the relatively short half-lives of the radioisotopes used in low dose radioisotope procedures it is preferable that the radioisotope seeds be sent to the hospitals just prior to their use. Because the number of radioisotope seeds varies from procedure to procedure depending upon the dose plan and because the cost of each low dose radioisotope seed is significant, it is not cost effective to order many more radioisotope seeds than will be used in a given procedure. Second, it is important to minimize the time of the procedure, both in terms of the exposure time of the physician to the low dose radioisotope seeds and in terms of the total time of the procedure from the economics of medical practice. The existing drop-in cartridge and seed magazine systems described above take longer to perform the implant procedure than using conventional preloaded implant needles because the radioisotope seeds are implanted one-by-one, rather than being delivered simultaneously as a group from a preloaded needle. Third, it has been routine to employ a radiophysicist at the hospital to preload the implant needles and take a set of sample measurements of the strength of the radioisotope seeds to confirm that the seeds meet the requirements specified by the dose plan. Finally, due to the large number of low dose radioisotope seeds used in a given procedure (typically up to 150) and the need for the implanting physician to be able to modify the dose plan at the time of implant, it is generally considered that the flexibility afforded by manually loading the implant needles just prior to the operation provides the best possible treatment procedure for the patient and the most economically efficient procedure for the hospital.

Although manual preloading of implant needles at the hospital continues to be the norm for most low dose radioisotope procedures, relatively little attention has been paid to increasing the safety or efficiency of this process. Presently, the radioisotope seeds for a given dose plan are shipped in bulk in a protective container to the hospital. At the hospital, the radioisotope seeds are dumped from the container onto a tray where the radiophysicist manually loads the seeds one-by-one into a set of implant needles according to the dose plan. Typically, the implant needles are positioned tip into a needle stand with the tips sealed with bone wax. The radiophysicist picks up a single radioisotope seed using a tweezers, forceps, or vacuum hose and deposits that seed in a needle. Next, a single spacer made of gut or similar absorbable material is deposited in the needle. This process is repeated depending upon the predetermined number of seeds and spacers prescribed by the dose plan. The radiophysicist will use a well chamber to measure the strength of a sample of the radioisotope seeds (typically from only one seed to a sample of about 10%).

Typically, the dose plan for a particular patient is generated by using one of a number of dose planning software programs in connection with an ultrasound probe to conduct what is known as a volume study. Examples of these programs include VariSeed™ from Varion Medical Systems, Prowess 3D® from SSGI/Prowess Systems, Interplant® from Burdette Medical Systems, and a system from Rossis Medical. Using these programs and the ultrasound probe, a physician essentially measures the size of the prostate gland. Based on the desired treatment in terms of both the level of radiation required for the treatment and the type of radioisotope seed to be used in the treatment, these programs recommend the number and placement of those seeds in order to accomplish the desired treatment. Typically, these recommendations are presented in terms of a dose plan grid that describes the starting plane relative to the base of the prostate gland at which the first seed should be implanted and the number of seeds to be implanted at that location. In some of these programs, the physician is allowed to adjust the recommendations by indicating locations on separate image planes generated by the ultrasound where additional seeds are to be added. Although these systems are an improvement over the previous manual calculation techniques for determining a dose plan, the output of these dose planning software systems are still only used in the manual process of loading needles as previously described.

More recently, systems that attempt to integrate the diagnostic process of establishing a dose plan using an ultrasound probe with a manual implant needle grid have been described in U.S. Pat. Nos. 5,871,448 and 6,129,670. In U.S. Pat. No. 5,871,448, a manual stepper arrangement for positioning the ultrasound probe is described. In U.S. Pat. No. 6,129,670, an automated arrangement is described for utilizing the ultrasound probe to generate ultrasound image data that is used to generate a translucent volume image of the patient's body and the prostate over which an image of the implant needles can be superimposed. The system develops a therapy plan for treatment of an organ by using an ultrasound probe to generate ultrasound data as is done with other dose planning systems. A device that provides a translucent volume image of a portion of the patient's body and a separate translucent image of the patient organ with a three-dimensional viewing capability to superimpose these two translucent images is also described. This patent also describes an automated system for loading radioisotope seeds into implant needles based on a clinical plan that enables rapid treatment based on substantially real-time preplanning using rapid patient organ evaluation. A gravity fed bin arrangement selectively drops seeds into the rear end of a vertically oriented needle. A pair of micro-controllers communicates with the computer that generated the dose plan to be the dose plan and control the dropping of the seeds and spacers into the rear end of the needle by using an optical sensor positioned along the passageway through which the seeds are dropped to monitor loading of each seed into the needle. The display on the microcontrollers of the autoloader displays to the operator a needle number, template coordinate location, and status of the needle being loaded. All of the needles are loaded in accordance with the predetermined dose plan, and there is no capability for the operator to modify the dose plan. Although the needle loading is proposed to be automated in this manner, the implantation of the loaded needles is accomplished manually using a conventional needle grid arrangement.

A modular device for implanting radioactive seeds through a needle implanted in the body is described in EP 1 070 519 A1. An electronic control device controls a pushing drive, a seed supply container, a spacer supply container and a multi-channel holder for seed-spacer trains. A tube connects the multi-channel holder and the needle through which the seed-spacer trains are pushed in order to implant them in the body. In one embodiment, the seed-spacer trains are loaded and implanted by a single unit. In another embodiment, the seed-spacer trains are preloaded into the multi-channel holder by a loading unit and then the multi-channel holder is then transferred to an implantation unit. In this embodiment, a microprocessor is used to control the seed loading unit in response to a therapy planning program. Like U.S. Pat. No. 6,129,670, the loading of seeds and spacers to form the seed-spacer trains in EP 1 070 519 A1 is accomplished directly in response to the therapy planning program executed that determines how the needles are to be placed in the prostate and how many radioactive seeds are to placed in what order in each of the needles. No provision is made for user control of the loading/implanting process. The only manner of interfacing with the system is the interface for the therapy planning program.

Other uses of automated arrangements for positioning ultrasound probes or for controlling biopsy needles have been proposed. U.S. Pat. Nos. 4,649,925, 5,181,514, 5,282,472, 5,361,768, 5,540,649 and 5,552,645 describe the use of automated arrangements for positioning of ultrasound probes. These automated arrangements typically include a stepper motor for advancing and retracting the ultrasound probe within the rectum and a rotational control for rotating the probe once in position within the rectum. U.S. Pat. Nos. 5,398,690, 5,415,169, and 5,830,219 describe automated biopsy arrangements in which a biopsy needle is inserted under automated control to obtain and extract a biopsy sample. These automated systems also include a single linear motion control and a rotational component control, and have an additional angulation control that controls the orientation of the needle upon insertion.

More complicated and expensive three-dimensional automated control systems for surgical instruments also have been developed. U.S. Pat. Nos. 5,540,649 and 5,695,500 describe examples of automated surgical systems that feature multiple joints and arms to allow for control of motion in all three axis of a surgical instrument positioned at the working end of these systems. The complexity and expense of these three-dimensional control systems have generally precluded their use in connection with positioning systems for ultrasound probes and biopsy needles.

All of the existing solutions for loading radioisotope seeds into implant needles rely on the user interface associated with the dose planning software to set out the way in which the radioisotope seeds will be loaded into the implant needles. It would be advantageous to provide for a system for automatically loading implant needles for low dose radioisotope procedures that could enhance the efficiency and flexibility of this process and provide additional features that are not possible with the current procedures.

### SUMMARY OF THE INVENTION

The present invention is a user interface for an automated radioisotope system. At least one source of a plurality of radioisotope seeds is provided that can be selectively ejected from at least one aperture of that source. A station is connected the source of radioisotope seeds. The station includes a computer processor that has a user interface that displays information about the automated system and accepts commands from a user to control the process of handling the radioisotope seeds. Preferably, this is accomplished by controlling an automated motion control system that selectively ejects radioisotope seeds from the at least one aperture in a replaceable cartridge that is the source of the radioisotope seeds. The station may be either a loading station for loading a plurality of implant needles that are to be used in a manual implant procedure, or an automated station for implanting radioisotope seeds as part of an automated implant procedure.

The computer processor in the automated system is preferably provided with a machine readable format of the predetermined dose plan. The user interface allows a user to alter a predetermined dose plan during the process of ejecting the radioisotope seeds from the cartridge. Preferably, the user interface is a touch-screen interface that displays a graphic representation of the coordinates of each location in the dose plan, with the user selecting the next needle, the next location to be processed by touching one of the coordinates. As the coordinate is touched, the icon associated with that coordinate changes color indicating that that needle had been processed. In addition, as each location is processed, a graphic representation of a cross-section of the needle is displayed to allow a user to confirm visually the proper arrangement of radioisotope seeds and spacers within the implant needle. For the loading station, the processing of each location corresponds to the loading of an implant needle for that location. For the implantation station, the processing of each location corresponds to the implanting of radioisotope seeds at that location.

In one embodiment, the user interface and computer processor create a transaction log that documents all of the edits and/or adds made to the dose plan during loading or implant procedures. Preferably, the computer processor communicates dose plan information to and from other devices via a standardized loading plan format. In one embodiment for a loading station, the user interface and computer processor can automatically adjust the number of spacers loaded in each needle so as to create a common zero retraction plane for all of the needles for a given patient. In one embodiment of the implantation station, the user interface and computer processor can automatically adjust the position of a base retraction plane in response to information from an ultrasound probe.

### BRIEF DESCRIPTION OF THE DRAWINGS

FIGS. 1A and 1B are perspective views of a preferred embodiment of the automated system for loading low dose radioisotope seeds and showing the preferred embodiment of the replaceable cartridge of the present invention in place within the automated loading system.

FIG. 2 is a perspective of the automated system of FIG. 1 with an enclosure and showing the receiving structure that mates with the replaceable cartridge of the preferred embodiment of the present invention.

FIGS. 3A and 3B are exploded perspective views of the preferred embodiment of the replaceable cartridge of FIG. 1 that loads needles from the rear.

FIG. 4 is a schematic representation of the various combinations of radioisotope seeds, spacers and plugs as stored in the rotatable drum of the preferred embodiment of the replaceable cartridge of FIG. 3.

FIG. 5 is a detailed view of a capstan assembly for the push rod of the preferred embodiment of the replaceable cartridge of FIG. 3.

FIG. 6 is a perspective of the assembled replaceable cartridge of FIG. 3 with a needle to be loaded from the rear.

FIG. 7 is an exploded perspective view of an alternative embodiment of the replaceable cartridge that loads needles from the tip.

FIG. 8 is a detailed cross-sectional view of a tip alignment structure, radiation sensor and needle sensing system of the replaceable cartridge of FIG. 9.

FIG. 9 is a perspective view of an assembled replaceable cartridge with a needle to be loaded from the tip.

FIGS. 10 and 11 are graphic depictions of a preferred embodiment of a user interface screen of a display of the automated system of FIG. 1.

FIGS. 12, 13, and 14 are graphic depictions of a dynamic fill feature of the user interface screen of a display of the automated system of FIG. 1.

FIG. 15 is a listing of a preferred file transfer format.

FIG. 16 is a detailed prospective view of the moveable assembly of the preferred embodiment of the present invention.

FIG. 17 is a perspective view of a preferred embodiment of the automated implantation station for implanting low dose radioisotope seeds and showing the preferred embodiment of the replaceable cartridge of the present invention in place within the automated implantation system.

FIG. 18 is a perspective of an alternate embodiment of the automated implantation station with an enclosure over the moveable assembly.

### DETAILED DESCRIPTION OF THE PREFERRED EMBODIMENT

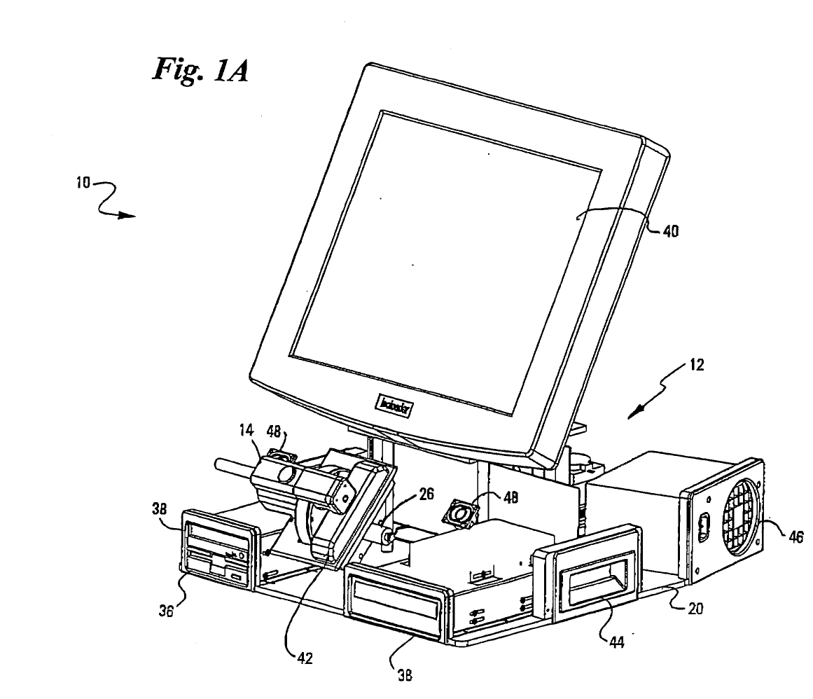

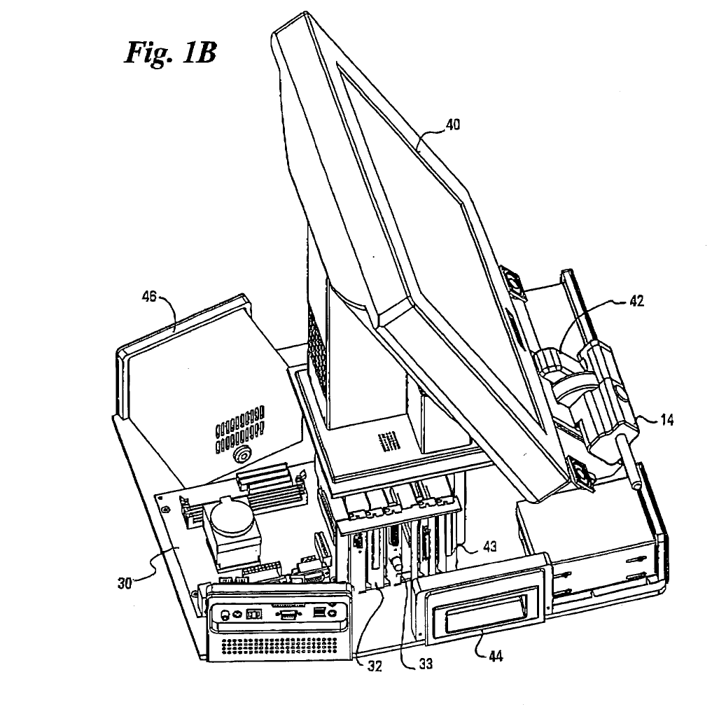

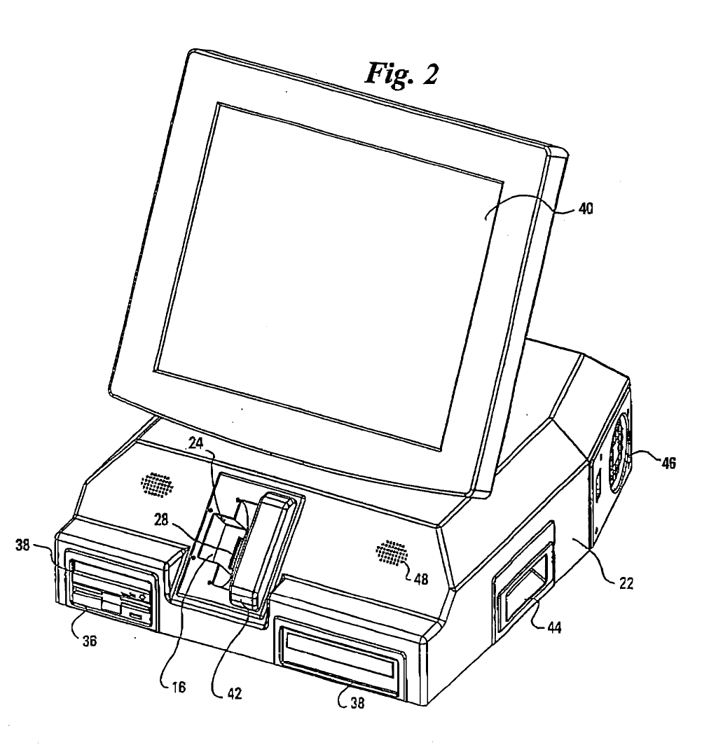

Referring to FIGS. 1A & 1B, an automated system 10 for loading low dose radioisotope seeds into a plurality of implant needles is comprised of a loading station 12 into which a replaceable cartridge 14 may be positioned. Preferably, the loading station 12 includes structure defining a cartridge receiving structure 16 in a front side of the loading station oriented toward a user as shown in FIG. 2. In this embodiment, the loading station 12 presents a front side toward a user with a corresponding longer dimension of the replaceable cartridge positioned in the cartridge receiving structure 16 parallel to this front side. Alternatively, the cartridge 14 and cartridge receiving structure 16 could be oriented transverse to the front side of loading station 12 or even at a rear side of loading station 12. The loading station 12 has a base 20 (as shown in FIG. 1) and a cover 22 (as shown in FIG. 2) preferably formed of molded plastic or metal. A computer processor 30 for the automated system is preferably a motherboard having a microprocessor, internal bus, a PCI-compatible bus, DRAM and EPROM or battery backed SRAM, with appropriate external interfaces or mated PC boards for a video interface, multiple channel IDE interfaces, a floppy disk interface, an ethernet interface, COM and LPT interfaces, an external bidirectional parallel port and a serial port. An automated motion control system 32 is preferably a Galil motion controller available from Galil Motion Control Inc. that interfaces to the computer processor 30 via the PCI-compatible bus. The automated motion control system 32 with appropriate software drivers provides all functionality for the lowest level control of stepper motor position and feedback sensors. A hard disc drive 34, floppy disk drive 36, high density removable media drive 37 and CD or CD-RW drive 38 are also provided for storing data and information to be used by the automated system 10. A video display 40 which operates as the primary user interface is preferably a 1280 by 1024 resolution flat 18.1 inch flat panel LCD with a resistive touch-screen, such as are available from National Display Systems. Alternatively, a conventional non-touch-screen video display and mouse, keyboard or similar input devices could also be provided. A proportional counter type radiation sensor 42 is positioned to be able to sense the passage of radioisotope seeds from the cartridge 14 into the implant needles and verify the radiation strength of the radioisotope seeds. In the preferred embodiment, the radiation sensor 42 is connected to a multi-channel analyzer card 43 that serves as a data acquisition device for information from this sensor. For clarity, none of the interconnections or cables among the various elements are shown in FIG. 1. FIG. 2 shows one of a pair of handles 44 for carrying the loading station 12 and one of two fan units 46 for cooling the circuitry and components of the loading station 12. Speakers 48 are also included in the front of the loading station 12.

Referring specifically to FIG. 2, the downwardly angled cartridge receiving structure 16 of the preferred embodiment will be described. The cartridge receiving structure 16 includes an angled channel 24 with sides that define a downwardly angled path of travel for inserting at a preferred angle of approximately 45 degrees. Once in position, the loading station 12 locks the cartridge in place using an electrical solenoid 26 (FIG. 1) to prevent inadvertent removal of the cartridge 14 during operation of the automated system 10. Locking is initiated automatically once the presence of a cartridge 14 has been detected in the cartridge receiving structure 16 and the user has initiated a loading operation via display 40. Unlocking the cartridge is initiated by the user selecting a remove cartridge operation via display 40, but only after computer processor 30 has confirmed completion of any critical motions that are part of the needle loading operation and removed power to the cartridge 14. Preferably, the only other interface between the cartridge 14 and the cartridge receiving structure 16 is a multiple pin-type electrical connector 28. As the stepper motors and associated encoder discs are contained within the cartridge 14, the need for extremely tight tolerance matches between the channel 24 of the cartridge receiving structure 16 and the cartridge 14 is minimized. In addition to the necessary control and sensor signals, the connector 28 includes a ground and power connection to provide power to the cartridge 14. The presence of cartridge 14 in cartridge receiving structure 16 is also detected via a contact on connector 28. Although an angled channel 24 is the preferred embodiment for interfacing the cartridge 14 with the cartridge receiving structure 16, it will be recognized that many other structures, such as guide rails, latches, pivoting arrangements, ball and detent locks, and orientations, such as horizontal or vertical, and connectors, such as optical, infrared, RF, slide contacts, array contacts or the like, could be used to accomplish the same function of interfacing the cartridge 14 with the cartridge receiving structure 16.

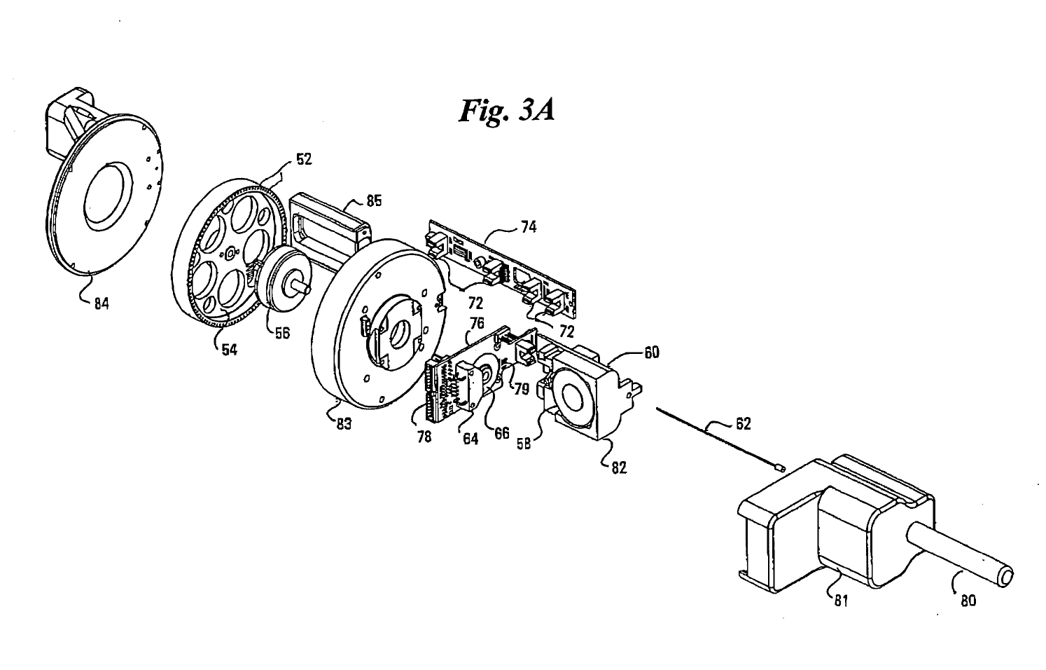

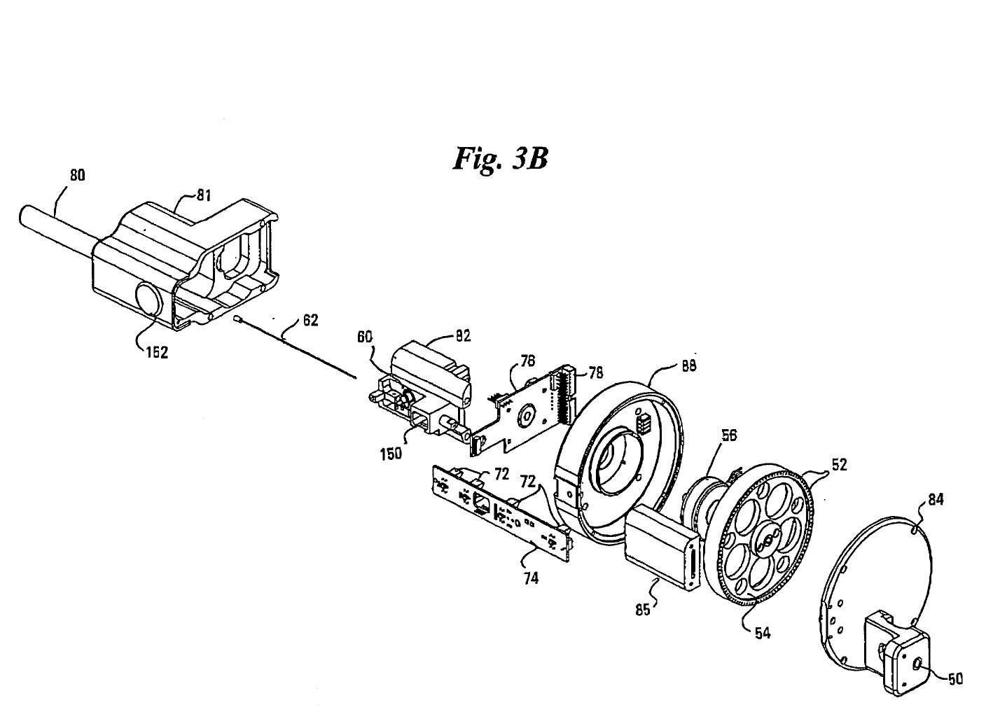

Referring now to FIGS. 3A and 3B, the cartridge 14 contains a plurality of radioisotope seeds and a plurality of spacers preloaded into the cartridge. The cartridge 14 has at least one aperture 50 into which an implant needle is positioned. Preferably, the radioisotope seeds and spacers are loaded into holes or chambers 52 located around the periphery of a rotatable drum 54. In this embodiment, the cartridge 14 includes a pair of stepper motors within the cartridge. A first stepper motor 56 rotates the rotatable drum 54. It will be seen that stepper motor 56 preferably drives rotatable drum 54 directly without any intervening gearing arrangement. A second stepper motor 58 has a capstan assembly 60 that rotates in engagement with a push rod 62 to slide the push rod 62. For the rotatable drum 54, an encoder detector 64 detects the position of a corresponding encoder disc 66 which is then communicated back to automated motion control system 32 (FIG. 1). Preferably, the stepper motor and encoder are selected such that the stepper motor steps in full steps with relation to the distance between chambers around the periphery. The alignment of the aperture to the chambers in the drum is preferably initially accomplished at the time of assembly. It will also be seen that other motor drives other than stepper motors could be used with equivalent success in the present invention, such as servo motors, worn driven motors, or DC motors with appropriate indexing control.

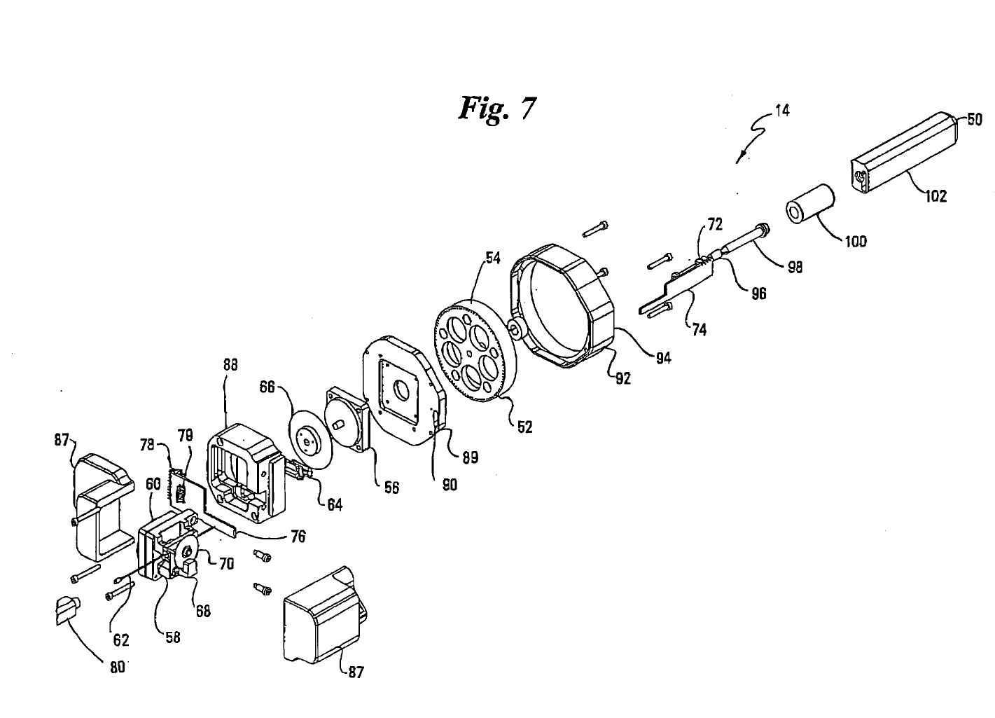

In an alternative embodiment as shown in FIG. 7, an encoder with a higher degree of resolution can be used and the stepper motor can be incremented in less than full steps. In this embodiment, a first encoder for the rotatable drum generates a positional feedback signal of an index of the chambers of the rotatable drum relative to the line of travel of the linear actuator 60, and a second encoder 68 with a second encoder disc 70 for the linear actuator 60 that generates a positional feedback signal of a position of the elongated member along the line of travel.

Referring again to FIG. 3, a series of position sensors 72 are positioned in line with the push rod 62 to detect the travel of push rod 62 as it is driven by capstan system 60 through its line of travel. The sensors 72 are connected to sensor circuitry 74 to communicate this position information to the automated motion control system 32. Each of the encoder detector 64 and sensor circuitry 74 are electrically connected to a circuit board 76 which has an appropriate connector 78 for mating with and connecting with a corresponding connector 28 (FIG. 2) in the cartridge receiving structure 16 of the housing 12.

Preferably, the circuit board 76 is provided with an electrically erasable programmable read-only memory (EEPROM) 79 or similar non-volatile memory to store parameters and other data that are unique to the particular cartridge 14 and to the particular patient and dose plan that has been developed for that patient. The contents of EEPROM 79 are set up initially during loading and calibration of the cartridge 14 at the factory. These contents are updated by the automated system 10 so as to continually reflect the current state of the cartridge 14. For example, when the radioisotope seeds and/or spacers are ejected from a given chamber 52, then the data on the EEPROM 79 is updated to reflect that the given chamber 52 no longer contains any radioisotope seeds and/or spacers. Preferably, the EEPROM 79 is capable of storing patient and hospital identification information, as well as seed inventory and manufacture information. Optionally, the EEPROM could also store the predetermined dose plan for the particular patient.

In the preferred embodiment, various housing elements enclose the cartridge 14 to create a single, enclosed drop-in cartridge to simplify operation and handling of the cartridge as shown in FIG. 3. Preferably, the various housing elements are formed of machined stainless steel to enhance the protective aspect of the housing. Alternatively, the housing could be formed of materials other than stainless steel. For example, the housing elements could be molded plastic with appropriate pieces having an internal lead lining or the like to provide sufficient shielding. Although the preferred embodiment is described as a single, enclosed drop-in cartridge, it will be understood by those skilled in the art that some or all of the functional components of cartridge 14 may be separately enclosed or left unenclosed and operably connected together to accomplish the same functionality, such as allowing for mating with the cartridge receiving structure 16 and protecting movement of the push rod 62 along its line of travel.

In the preferred embodiment of the rear loading cartridge 14 as shown in FIG. 3, a push rod sleeve 80 encloses the travel of push rod 62. Cover 81 is a one-piece unit that covers the capstan assembly 60 and its associated components. A capstan motor mount 82 provides a mounting base for most of the main components of cartridge 14, including circuit board 76 and encoder detector 64. Housing 83 houses the stepper motor 56 and the rotatable drum 54. A cover plate 84 mounts to the housing 83. The motor mount 82 and the cover 81 are secured by internal screws (not shown) that are accessed when the cover plate 84 is removed. A front plate 85 covers the circuit board 76 and is also mounted with screws between cover plate 84 and cover 81. A needle housing 86 is also screwed on to the cover plate 84 and includes the aperture 50 through which the needle accesses the cartridge.

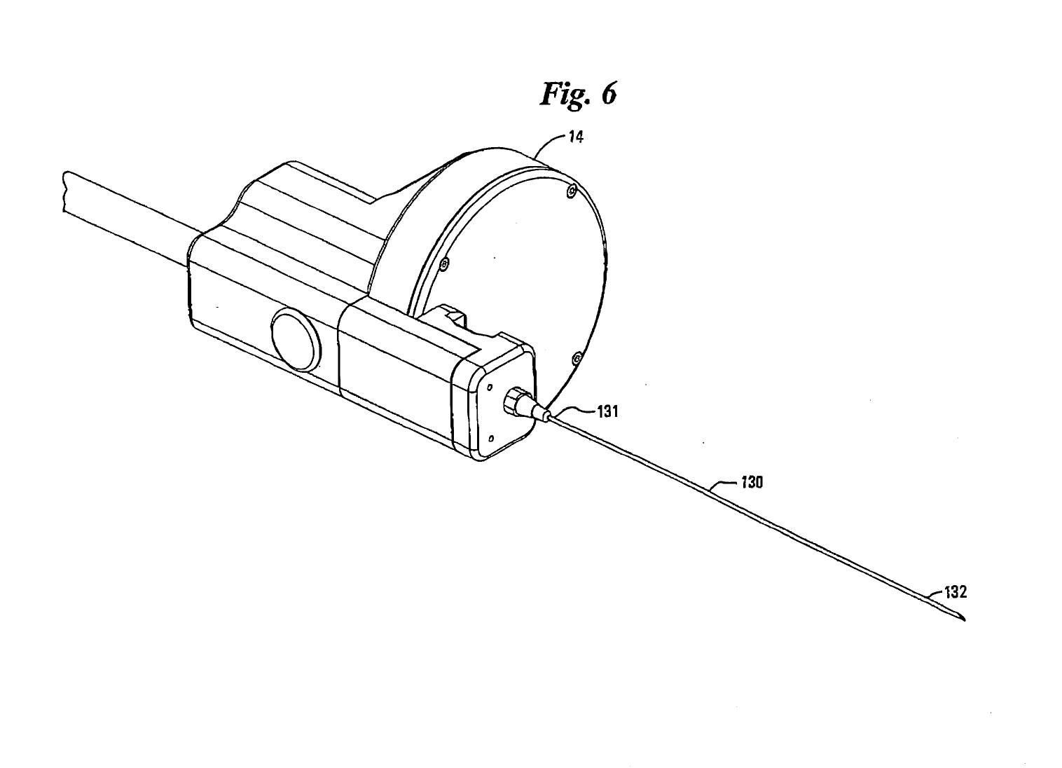

In the preferred embodiment as shown in FIG. 6, the contents are loaded into the rear 131 of the implant needle 130 which has its tip 132 plugged with bone wax or a similar plug material. Alternatively, a crimp at the tip 132 could prevent the contents of the chamber from being pushed out of the tip 132 of the needle 130 as it is loaded from the rear 131. In this embodiment, the rear 131 of the needle 130 is preferably secured in place in the aperture 50 by a Luer lock or similar assembly. Preferably, the tip 132 does not extend beyond the side of loading station 12 as a safety measure.

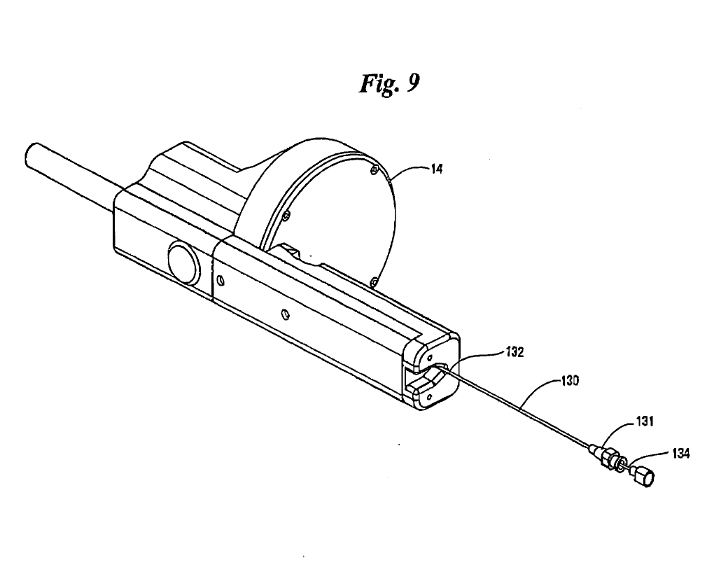

In an alternate embodiment as shown in FIGS. 7 and 9, the contents are loaded into the tip 132 of the needle 130, rather than into the rear 131 of the needle 130. In this embodiment, the housing elements are configured somewhat differently than in the rear loading embodiment. A rod sleeve 80 encloses the travel of push rod 62. Housing halves 87 mate to a base 88 to cover the capstan assembly/linear actuator 60 and its associated components. The base 88 provides a mounting base for most of the main components of cartridge 14 of the tip loading embodiment, including circuit board 76 and encoder detector 64. Plate 89 provides a mounting structure for stepper motor 56 and includes an aperture 90 through which push rod 62 slides to engage the radioisotope seeds and spacers located in the chambers 52 around the periphery of rotatable drum 54. Plate 89 also prevents radioisotope seeds and spacers from falling out of the chambers 52 on one side of rotatable drum 54. A cap-like cover 92 is mounted over the other side of rotatable drum 54 and includes an aperture 94 by which access is provided to sensor circuitry 74 and through which push rod 62 slides to eject the radioisotope seeds and spacers into the implant needle (not shown) via an alignment tube 96. An alignment structure 98 preferably comprising a beveled alignment needle guide has an internal channel that aligns a corresponding beveled implant needle with the alignment tube 96. An electrical solenoid 100 is used to lock the implant needle in place relative to the cartridge 14 once the proper positioning of the implant needle in the alignment structure 98 has been confirmed. In this embodiment, the at least one aperture 50 is defined on an end of a shield tube 102 constructed of appropriate metal to shield the radioisotopes as they are being loaded into the implant needle.

In addition to the advantages afforded by constructing cartridge 14 as a single, enclosed drop-in cartridge, the preferred embodiment of cartridge 14 is designed with minimum piece parts to allow for easy disassembly and sterilization to allow for potential re-use. Once the various covers and circuit assemblies are removed, the remaining portions of cartridge 14 are cleaned with alcohol or hydrogen peroxide to remove bioburden. When reassembled, the entire cartridge 14 is preferably sterilized with a gas sterilization technique. The ease of disassembly also provides a convenient mechanism by which emergency removal of the radioisotope seeds can be accomplished, simply be removing cover 92 and dumping the radioisotope seeds and spacers into an appropriate container.

The use of a rotatable drum 54 also affords important advantages to the preferred embodiment of the present invention. The positioning of the chambers 52 around the periphery of drum 54 reduces the concentration of radiation sources at any given point and provides an optimum separation of radioisotope seeds from each other, thereby enhancing the safety of cartridge 14.

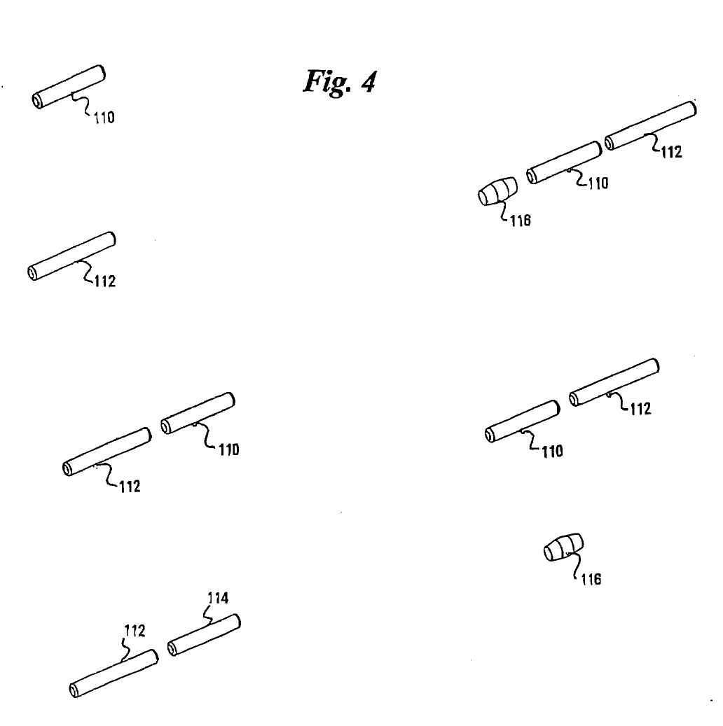

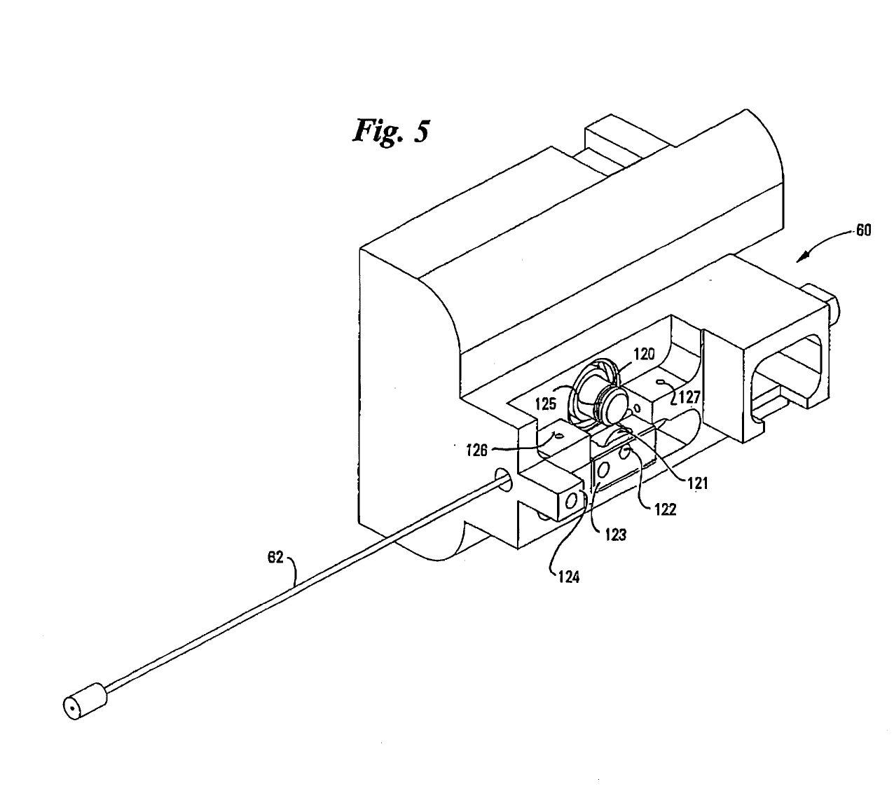

In the preferred embodiment, each chamber 52 is long enough to accommodate any of a combinatorial set of radioisotope seeds, spacers and plugs. As shown in FIG. 4, various combinations of radioisotope seeds 110, full-length spacers 112, partial-length spacers 114 which can serve as blanks, and plugs 116 can be positioned within a given chamber 52. In this embodiment, the length of one radioisotope seed 110 or one blank 114 is 4.5 mm, the length of one full length spacer 112 is 5.5 mm and the length of one plug 116 is 2 mm. As will be apparent, the selection of the lengths of each of the seeds 110, spacers 112, 114, and plugs 116 allows for various combinations to be utilized that have the same overall length when positioned in an implant needle of 10 mm for seed and spacer or 12 mm for seed, spacer, and plug. The particular combination of each for a given cartridge is optimally determined at the time that the cartridge 14 is preloaded in accordance with a predetermined dose plan. This information can then be utilized by the automated station 10 to load the implant needles in accordance with that predetermined dose plan.

In the preferred embodiment, the rotatable drum 54 is provided with 200 chambers 52 spaced equidistant about the periphery of the rotatable drum 54. The optical encoder disc 66 preferably has 400 or 1600 lines of resolutions which yields a resolution of 2 or 8 counts per chamber 52. In an alternate embodiment with higher resolution as previously described, 72,000 lines of resolution are used which yields a resolution of 360 counts per chamber 52. A home reference is provided by an index channel on the encoder disc 66. The alignment of the aperture 50 to the chambers 52 in the drum 54 using the index channel is preferably accomplished at the time of assembly. In the high resolution embodiment, an offset to a first chamber location clockwise from the home reference is stored as a parameter for the cartridge 14 to allow for individual cartridge tolerance calibration. Alternatively, an optical sensor could be used to locate the center of a chamber 52 for purposes of calibrating an index. In operation, the automated motion control system 32 uses the stepper motor 56 and encoder detector 64 to establish a reference to the first seed drum chamber 52. Motion of the drum 54 may take place bidirectionally (i.e., clockwise or counterclockwise) and as rapidly as possible in order to move to the nearest desired chamber location as determined by the computer processor 30 and automated motion control system 32 in the shortest possible time. When requested by the computer processor 30, the automated motion control system 32 will index to the center of the desired chamber location in preparation for transfer of the contents of that chamber 52 to the implant needle. The drum 54 will remain at this location until it is commanded to a new position.

A positive travel limit is preferably established using a first optical sensor 126 that is part of the structure of capstan assembly 60 which detects the back of the push rod 62 passing through a defined point. A negative travel limit for the line of travel of push rod 62 is established by a second optical sensor 127 that doubles as a home reference. Preferably, the travel limits do not disable the stepper motor 58, but rather send an indication to the automated motion control system 32 that the respective travel limit has been exceeded. Once zeroed in relation to the home reference, the push rod 62 is moved forward and into an open chamber 52 in the drum 54. This serves as a loose mechanical lock to prevent the drum 54 from being rotated unintentionally. When a request for a seed transfer is generated by the computer processor 30, the automated motion control system 32 activates the capstan assembly 60 to retract the push rod 62, thereby allowing the drum 54 to be rotated freely.

When the drum 54 has been indexed to the desired chamber location, the automated motion control system 32 instructs the stepper motor 58 to move the push rod 62 forward to push the contents of the chamber 52 out of the drum 54 and into the tube 96 leading to the radiation sensor 42. The distance the push rod will travel will be based on the total length of the contents in the given chamber and the location of the radiation sensor 42. Because the automated motion control system 32 knows the nature of the contents of each chamber 52, the push rod 62 would be instructed to stop and position the radioisotope seed in front of the radiation sensor 42 if a radioisotope seed was present in the contents of a given chamber and if the computer processor 30 determined that a radiation measurement should be acquired based upon the radiation sensing parameters as set by the user of the automated system 10. In this case, a message would be communicated from the automated motion control system 32 to the computer processor 30 when the radioisotope seed 110 was properly positioned indicating that a radiation measurement may be performed. Once a radiation measurement has been taken, or if no radiation measurement is required, the automated motion control system instructs the stepper motor 58 to move the push rod 62 forward to deliver the contents into the implant needle 130.

The trailing one of the position sensors 72 is provided along the path of material transfer to allow for detection of the leading edge of the contents with relation to the tip of push rod 62. As the contents of a given chamber 52 are moved by the position sensor 72, the total length of the contents may be determined. This allows for a verification of the length of the contents of a given chamber 52 with the information the automated system has about what should be in that chamber 52 to prevent potential misloads. In the event of an early or late activation of the sensor 72 by the tip of the push rod 62 in relation to the expected activation based on the anticipated length of the contents of that given chamber 52, an alarm or error message would be passed to the computer processor 30.

In the tip loading embodiment as shown in FIG. 9, as the contents are delivered into the implant needle 130, a stylet 134 that is preferably positioned in the implant needle 130 is pushed back by the advancing contents. In this way the needle 130 and stylet 134 are ready to use as soon as the loading process is completed and it is not necessary to insert a stylet into the implant needle after the loading process is completed, thereby incurring the risk that the stylet would dislodge the plug 116 or displace any of the loaded contents from the implant needle 130.

As any given implant needle 130 may be loaded from the contents of one or more chambers 52, it is important that the contents of a given chamber 52 containing a plug to be inserted at the tip 132 of implant needle 130 be accurately aligned with the end of the tip 132. In this case, the automated motion control system 32 preferably moves the contents of the chamber 52 containing a plug to an absolute location relative to the tip 132 of the implant needle 130, rather than moving the contents a relative distance based on the expected lengths of the contents of that chamber. In this way, the plugs 116 are always inserted so that they are flush with the ends of the tips 132 of the implant needles 130.

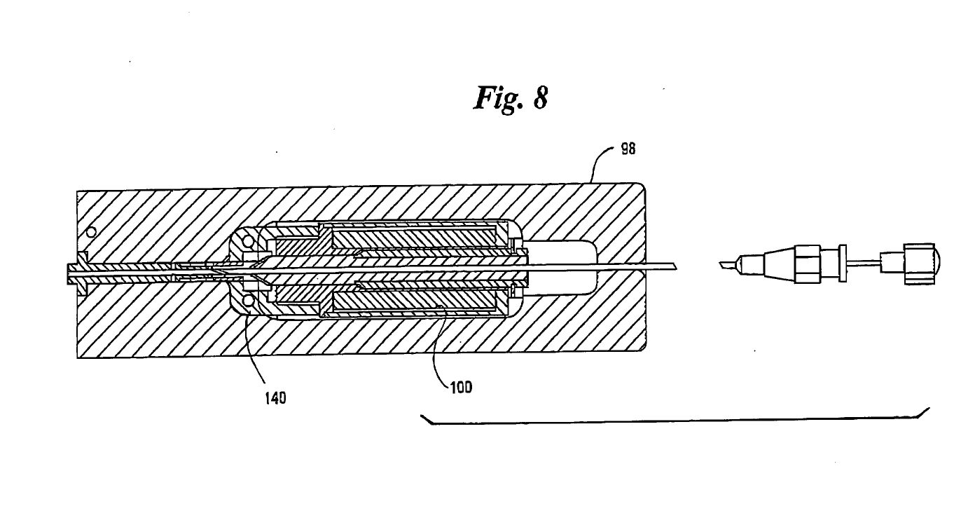

Referring now to FIG. 8, an embodiment of the alignment structure 98 and the positioning of an implant needle 130 will be described. In order to begin a loading cycle, the needle tip 132 must be properly positioned by the user so that a known location is established for the needle tip 132. An optical sensor 140 is positioned precisely at the desired location of the needle tip 132 and is connected to the sensor circuitry 74 (FIG. 1). Preferably, the alignment structure 98 is beveled to match a beveling on the tip 132 of the implant needle 130. To accomplish proper alignment, the user inserts the implant needle 130 into the aperture 50 until it abuts alignment structure 98 and then rotates the implant needle 130 until the optical sensor 140 indicates proper alignment. Preferably, the optical sensor 140 remains active during the loading process to confirm that there is no movement of implant needle 130 during this process. Once the proper positioning of the implant needle 130 has been confirmed, an electrical solenoid 100 is activated to clamp the implant needle 130 in place relative to the cartridge 14. The force of the solenoid 100 is such that the implant needle 130 may not be moved during the loading operation, but not sufficient to crush the implant needle 130. In the preferred embodiment, the solenoid 100 is automatically released once the loading of the implant needle 130 is complete and a plug 116 has been inserted into the tip 132 of the implant needle 130.

Although the cartridge 14 of the present invention has been described with respect to the automated station 10, it will be understood that the cartridge 14 of the present invention may also be used with other automated equipment as part of a low dose brachytherapy procedure. For example, the elongated member used to eject the radioisotope seeds in the preferred embodiment is a push rod 62 that loads the seeds into a plurality of implant needles. Where the cartridge 14 is used with an automated needle insertion system, the elongated member may be a trochar needle or similar cutting member that would first make an incision into the patient, then be withdrawn, and finally advanced through the aperture of the cartridge to eject the seeds.

Although the drum 54 has been described as the preferred embodiment of the positional member of the cartridge 14 with its movement controlled by stepper motor 56, it should be understood that other forms of this positional member and other motor arrangements would also work within the scope of the present invention. For example, the positionable member could be an X-Y grid of chambers with a pair of stepper motors used to drive the grid in X-Y directions to position the desired chamber in line with the aperture and push rod. 62. Although stepper motors, such as stepper motor 56, and encoders, such as encoder 64 are a convenient and economical manner of implementing the present invention so that it may be controlled by an external microprocessor arrangement, it will be recognized that other arrangements such as gears, drive belts and clutched motor shafts could be used in place of the stepper motor, and that contact sensors, optical sensors or registry from a known starting point could also be used in place of the encoder. It will also be seen that while the preferred embodiment interfaces with an external microprocessor, it would also be possible to incorporate a microprocessor into the cartridge itself and to communicate externally by telecommunications, radio communications or the like, instead of by electrical connectors.

For a more detailed description of the preferred embodiment of the radioisotope seed cartridge 14 and its preferred operation and loading, reference is made to the previously identified co-pending application entitled “RADIOISOTOPE SEED CARTRIDGE.”

In the preferred embodiment of the loading station, radiation in the form of x-rays from the radioisotope seeds 110 is detected by a radiation sensor 42 that is a LND zenon-filled proportional counter tube. This tube outputs pulses at a rate that is determined directly by the frequency of decay events and the pulse height is determined by the energy of the individual photons associated with each decay event. To quantify the radiation activity of a given source, all of the pulses having a height within a given band of interest are counted for a predetermined period and the rate is compared to a known reference. It will be understood that the particular requirements for positioning of a radioisotope seed 110 in front of the radiation sensor 42, such as positional tolerances or dwell time required for adequate measurement, may be different for different radiation sensors, and that trade-offs between the time required for radiation sensor readings and the accuracy of those readings may be made. Alternatively, it may be possible for certain radiation sensors 42 to take measurements while the radioisotope seeds 110 are moving by the radiation sensor 42, either at a normal rate of travel or perhaps at a reduced rate of travel. In another embodiment, the push rod 62 is instructed to stop or slow down in front of the radiation sensor 42 for each item in the contents of the chamber 52 to verify that the contents are as expected (e.g., a spacer 112 registers no reading and a radioisotope seed 110 registers a reading). This type of verification can be quick and simple and would not require a complete characterization of the output of radiation sensor 42.

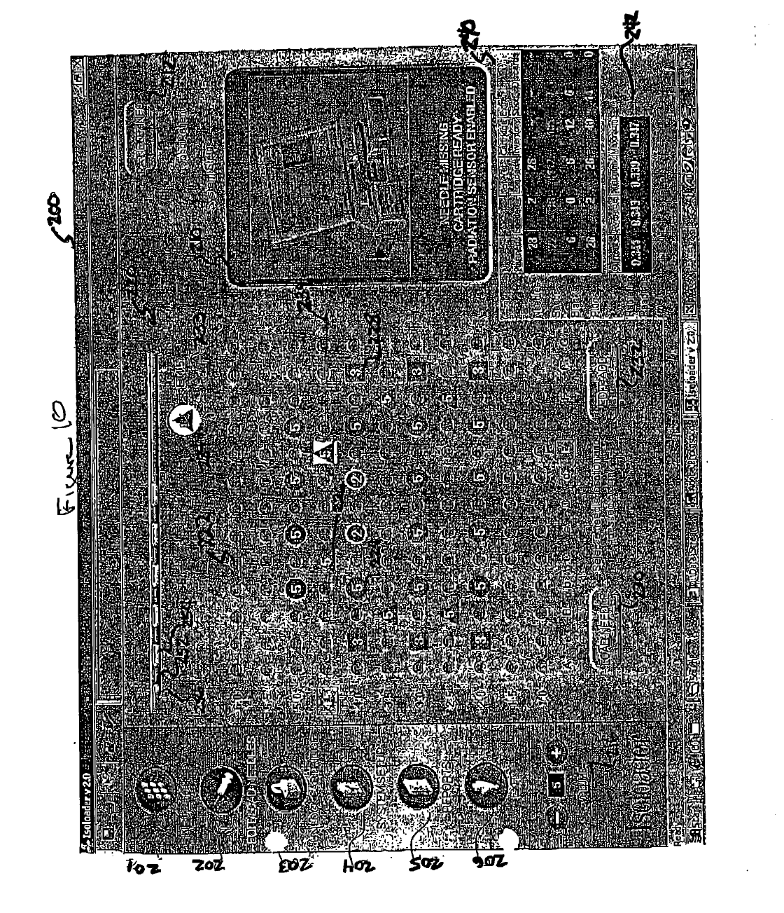

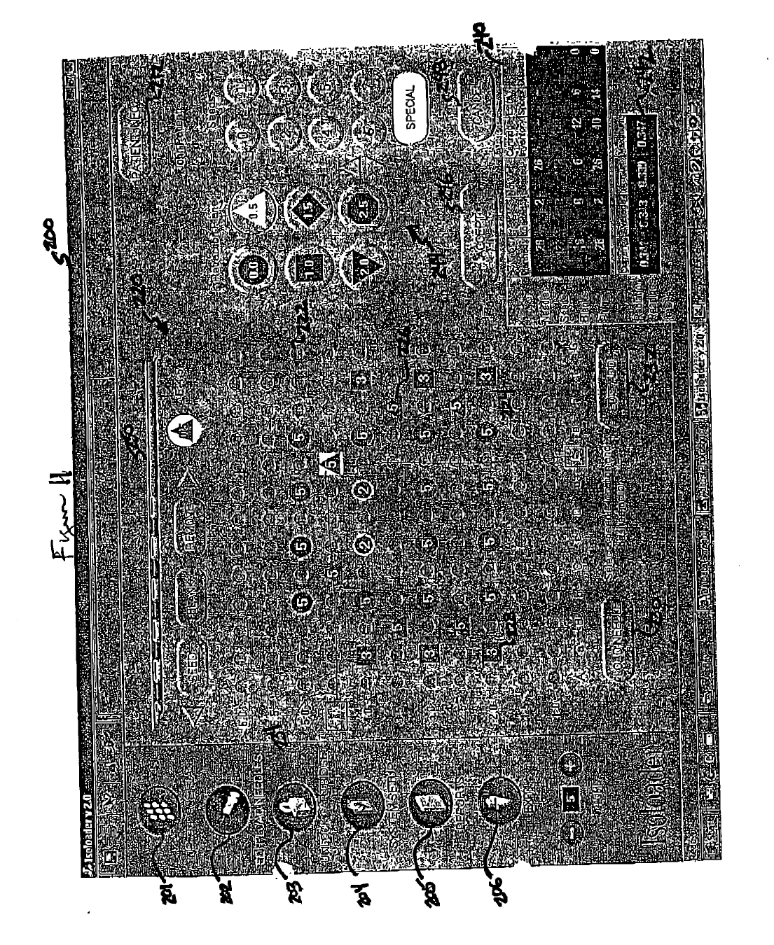

Referring now to FIGS. 10 and 11, a preferred embodiment of the user interface 200 as presented on display 40 (FIG. 1) will now be described. Preferably, the display 40 is a touch-screen display and the computer processor 30 utilizes a Windows® NT operating system with a Radisys® In Time environment. To a user, however, the user interface 200 preferably appears as a dedicated virtual machine having a single primary touch-screen user screen as shown in FIG. 10. Although the preferred embodiment of the present invention will be described in connection with a touch-screen user interface 200, it will be recognized that various other user interfaces, such as conventional video displays, LCD displays or specialized displays may also be used with the present invention. In addition, it would be possible to provide for an audio-controlled user interface coupled with an optional display screen to allow for voice-activated control of the loading process.

In the preferred embodiment of user interface 200, a series of dedicated touch-activated buttons 201 to 206 are positioned to always remain visible on the left side of the display. The user interface 200 is preferably designed to provide a very flat icon-based menu structure with minimal overlay windows where all of the functions controlled by a user are accessible through each touch-screen inputs. A virtual keyboard may be selected to enter alphanumeric data. Alternatively, a mouse and keyboard may be connected to the computer processor 30 to enter such data. Another equivalent input device is a joy stick or game port pad or equivalent pointing/directional input device. Preferably, each of the buttons 201-206 has an icon on the top half of the button and a corresponding text message on the bottom half of the button. A status icon 210 is preferably displayed along the left of user interface 200 to display status messages such as Cartridge Detected, Reading Inventory, Running Diagnostics, Verifying Radiation Sensors, Cartridge Ready, Printing, and the like. Once a cartridge 14 has been successfully loaded and locked into the cartridge receiving structure 16, at least the patient name information from the EEPROM 79 of that cartridge 14 is displayed in the top left corner of the user interface 200. Additional patient information can be accessed through button 212. In a preferred embodiment, the system status area 210 is also used as a multi-media help screen that can display information about using the system 10, as well as general information about the particular brachytherapy procedure to be performed. A volume control 216 is provided to conveniently control the audio volume of multi-media information displayed on the status area 210.

The primary display in the main part of the user display 200 is the loading pattern grid 220 which replicates an interactive grid of how the implant needles 130 are to be loaded in a format that is similar to the paper format currently used for prostate cancer brachytherapy procedures. In this format, the numbers along the left side of grid 220 represent the height in centimeters and the letters represent the width in 0.5 centimeter increments (1.0 centimeters between capital letters) of the locations where the implant needles 130 are to be inserted from a reference base axis that would be located at 0.0. The open circle icons 222 at the intersection of each of these coordinates represents a chamber in an implant grid that is used to implant the series of implant needles 130. Each of the icons 224, 226, 228 in the center of grid 220 represent an implant needle 130 with the number in the center of the icons 224, 226, 228 indicating the number of radioisotope seeds 110 that are planned for that implant needle 130. The icons 224 are for needles in which the seeds 110 are spaced at regular intervals using full-length spacers 112. The icons 226 are for needles in which the seeds 110 are spaced at regular intervals, but are offset or staggered by using at least one partial-length spacer 114. Icons 228 represent those needles in which the seeds 110 are not spaced at regular intervals due to the staggering of partial length spacers 114 and full length spacers 112.

The grid 220 is active, as shown in FIG. 11, when the Edit/Add Needles button 232 is activated. The currently active location is indicated by the message 233 at the upper right corner of the grid 220 and by the intersecting lines 234 that highlight that coordinate in the grid. A user selects a different currently active needle location by pointing to that location. In one embodiment, the status of each of the icons 224, 226, and 228 are conveniently shown in the colors as indicated in the scoreboard area 240. The scoreboard area 240 is dynamically updated by the computer 30 to reflect the planned, loaded, not yet loaded, cartridge inventory, extras and discards that the user has available or has used. A radiation reading area 242 displays the information generated by radiation sensor 42. The Edit control area 244 allows a user to select retraction plane depths and number of seeds for the active needle location. Once the desired configuration is selected, the user accepts the configuration for the active needle location by entering button 246. Alternatively, the information for this location can be discarded by selecting the cancel button 248.

Once a user activates the Load Needle button 230 as shown in FIG. 10, the user is instructed to insert an implant needle to be loaded by the system status message 210 at the right of the user interface 200. When an implant needle 130 is detected in the aperture 50, an needle needle icon 250 representing the needle 130 is displayed at the top of the user interface 200. In the tip loading embodiment, this icon is interactive in response to the orientation and alignment of needle 130 as detected by optical sensor 140 as previously described. For example, the orientation of the beveled end 256 of needle icon 250 could rotate until alignment was achieved, at which time the color of the needle icon 250 would change from a red background to a green background and a text message in the system status area 210 that the needle was present and locked would also be displayed. As the implant needle 130 is being loaded, position indicators 252 and 254 in the needle icon 250 represent locations in the implant needle in which radioisotopes 110 and spacers 112, 114 may be loaded. As the loading process progresses, seed icons 252 and spacer icons 254 are displayed in the respective position indicators where those items are positioned in the implant needle 130. In the case of the tip loading embodiment, once a plug 116 is inserted at the tip 132 of implant needle 130, a plug icon 256 is displayed at the end position indicator and the needle icon 250 would change to a white background while the system status area 210 would be changed to indicate that the implant needle 130 was now loaded and could be removed. At this point, the computer processor 30 would instruct the solenoid 100 to unlock the implant needle 130.

The Input Dose Plan button 201 allows a user to input a predetermined dose plan. Two input options are provided, a Manual Input option and a Load File option. In the Manual Input option, the grid 220 is displayed with no predetermined dose plan overlaid. In this mode, the user would select a desired location and then use the Edit/Load Needle button 202 to indicate how the implant needle 130 corresponding to that location should be filled. This process would then be repeated for each implant needle to be loaded via this manual option. In the Load File option, a pop-up window is displayed showing the default dose plan that was used to generate the configuration of contents of the particular cartridge 14. In a preferred embodiment, a compact disc (CD) is delivered along with the cartridge 14 to the hospital where the procedure is to be performed and the default dose plan is contained on this CD and is read by the CD player 38. In another embodiment, a compressed version of the default dose plan is stored on the EEPROM 79 in the cartridge 14. If the automated system 10 was used during the generation of the dose plan at an initial planning visit or at the time of the procedure, then the dose plan would be stored on the hard drive 34. Alternatively, the default dose plan could be stored on a floppy disc and read by the floppy disc drive 36 or could even be stored on a remote location and accessed by an external interface, such as by an encoded transmission over the Internet or over a private dial-up network. If the user desires to override the default dose plan and select another dose plan, the pop-up window would allow the user to search the various drives accessible by the automated station to locate an appropriate dose plan file. Preferably, the default dose plan is stored in a proprietary text file format adapted for use by the software running on the computer processor 30. Alternatively, the computer processor 30 could translate the output files of any of a number of dose planning software packages to the proprietary text file format as part of the process of loading the dose plan. Once an appropriate file has been selected, the user can load the selected file as the dose plan and the details of that dose plan are then displayed on the user interface 200. Alternatively, the computer processor 30 could be provided with the dosimetry software package and a user could develop the dose plan directly on the computer processor 30 either prior to the procedure or during the procedure. For example, the dose plan could be modified as the procedure progresses in response to needles that have been loaded. In this embodiment, a common file structure could be shared between the dosimetry software and the control software running on the computer processor 30 for controlling loading of the needle 130.

The Unlock Cartridge button 203 is used to instruct the automated system to initiate the process of preparing for the cartridge 14 to be removed from the cartridge receiving structure 16. Various checks are performed by the computer processor 30 to insure that certain tasks are completed. These tasks include confirmation that no implant needles are in the cartridge, a verification that the current inventory of the seeds 110 in the drum 54 is stored in EEPROM 79, a homing function for the push rod 62 into an empty chamber 52 in drum 54 to lock the drum 54 into position. After these tasks are completed, power would be shut off to the cartridge 14 and the solenoid 26 is deactivated to unlock the cartridge. A pop-up message is displayed to the user instructing them to manually remove the cartridge 14 from the cartridge receiving structure 16 and providing for an option to cancel this operation. Preferably, a countdown timer is shown during which time the user would be able to manually remove the cartridge 14 and after which the solenoid 26 would be engaged again to relock the cartridge 14 in place. The contact on the electrical connector 28 is monitored to confirm that the cartridge 14 has been removed and the pop-up windows are closed once the cartridge 14 has been removed.

The System Setting button 204 allows the user to view and edit various parameters of the automated system 10, including radiation measurement parameters, radiation calibration settings, motion control parameters and display preferences. In the case of radiation measurement parameters, the user is preferably given the option in a set-up window of choosing to monitor (i) all contents, (ii) all seeds, (iii) every given number of seeds, or (iv) only the first seed in each implant needle. Optionally, the estimated time required to load an average implant needle at each setting can also be displayed. The radiation calibration settings would also have a set-up window that would take a user through the process of testing the radiation sensor 42 by inserting a radiation source of a known intensity into the aperture 50 and positioning that source in front of the radiation sensor 42.

The Reports button 205 allows the user to print out certain predetermined reports for the automated system 10, including a loading plan report, a radiation reading/calibration report, a case summary and a system diagnostic report. These reports may be printed directly over the external connections for computer processor 30, may be stored to a file for later printing or review. The user may be provided with certain formatting preferences and printing options to customize certain details of the presentation of these reports.

The Exit button 206 allows the user to exit or switch from the needle loading application software back to the operating system software running on the computer processor 30. This button 206 can either be conditioned on a proper shutting down of the automated system 10, including removal of the cartridge 14, or it can allow for an option to switch to another application that could be running on computer processor 30. In one embodiment of the present invention, the computer processor 30 is provided with dose planning software that would be used by the physician to create the predetermined dose plan that is to be used by the needle loading application software.

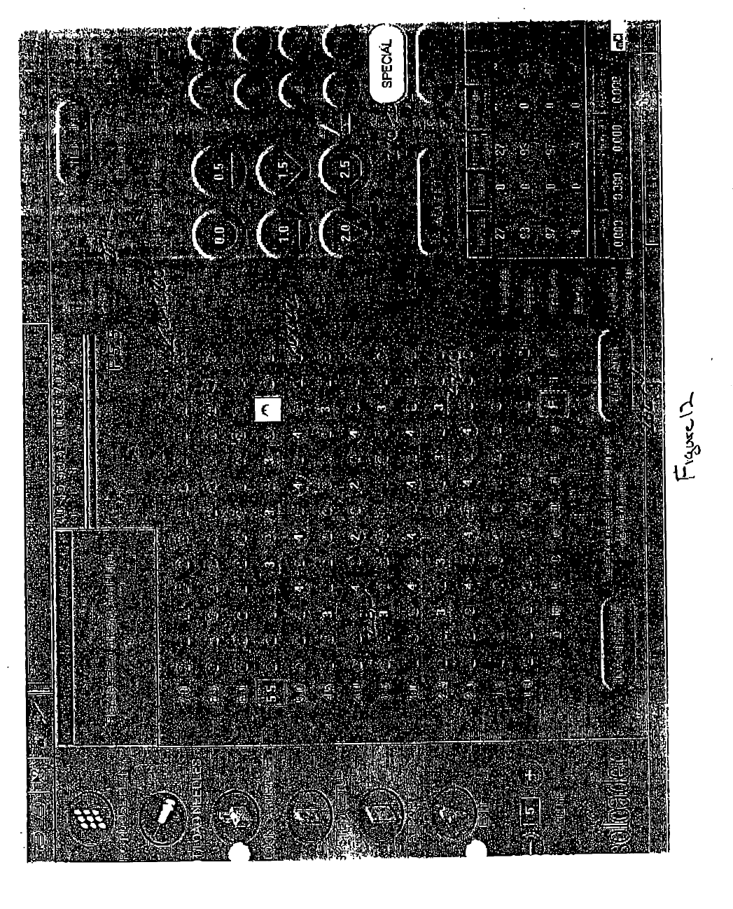

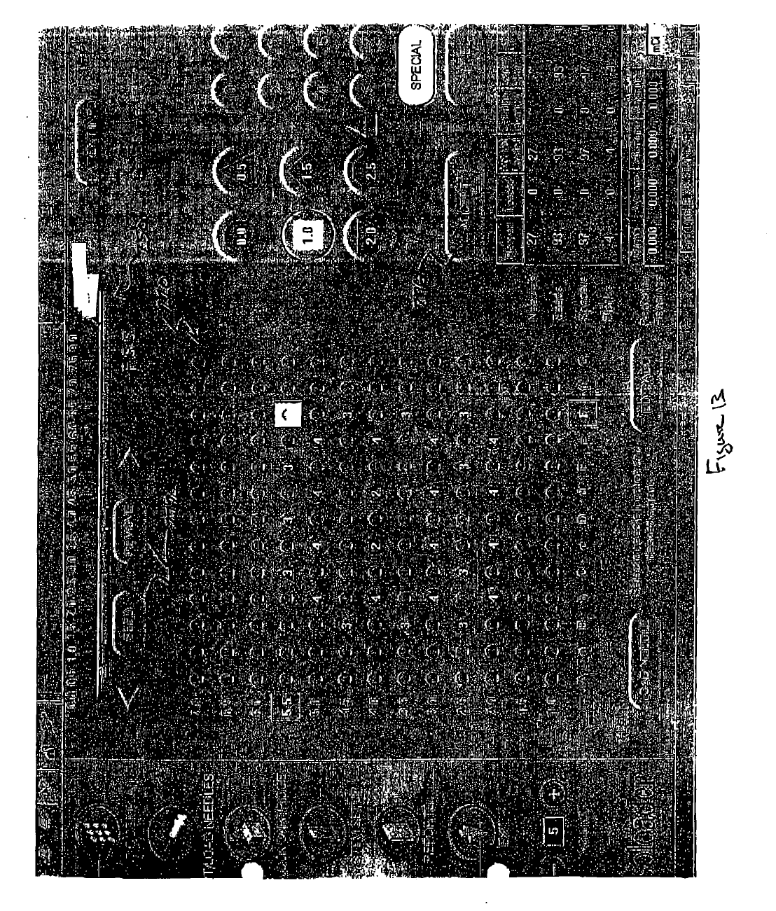

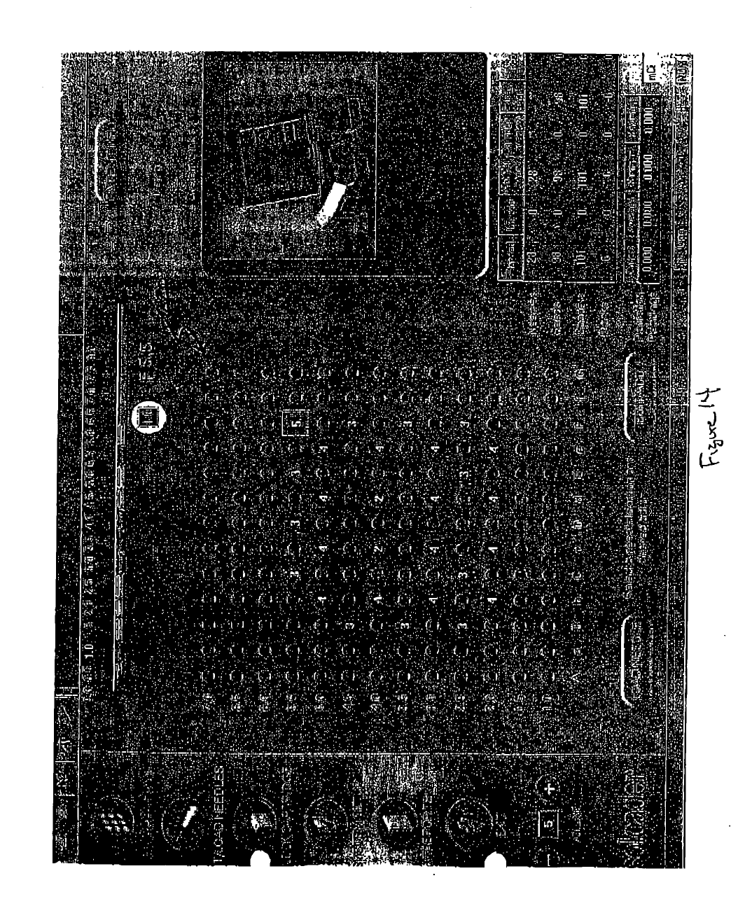

In one embodiment as shown in FIGS. 12, 13, and 14, a special loading mode with dynamic fill in is provided. In this embodiment, a user would select an icon 224, 226, or 228 from the grid 220 representing a needle 130 to be loaded. When the edit button 232 is chosen, a special mode button 270 can be selected which activates an interactive version 272 of the needle needle icon 250. In this interactive version 272, a user can indicate where along the needle icon the user would like to have seeds located. As shown in FIG. 12, the special mode button 270 is pressed and the user is prompted to select a retraction plane 274. The retraction plane 274 is defined as a plane in the location of the middle of the initial seed to be loaded/implanted for that location. FIG. 13 shows how a user then places additional seeds at locations as desired along the interactive version 272. As will be seen, in a preferred embodiment there is no need for the user to place spacers in this embodiment as this is done automatically. As shown in FIG. 14, the computer processor automatically fills in the spacers in appropriate locations for the positions indicated by the user. Finally, after selecting an accept button 276, the pattern of seeds, spacers, and blanks as shown in interactive version 272 is accepted and added to the dose plan. Preferably, the resolution of available locations along the interactive version of the interactive version 272 is 0.5 mm.

In one embodiment, the computer processor 30 creates a transaction log of all events that occur during the loading of the needles. The transaction log can be stored in memory for subsequent redisplay or transmission to another computer, copied to removable storage such as a CD or floppy disk, or printed out to provide a written record of the needle loading process. The transaction log is also used for the purposes of integrating data from the transaction log with report generating software to generate customized reports for the procedure. Such custom reports would include, for example, patient identification information, identification information for the particular batch of radioisotope seeds, total number of seeds loaded and average activity per seed. Details such as a complete map or plan of where each seed was loaded can also be incorporated into a customized report or can be generated separately and attached to such a report. The transaction log is generated during the loading and editing process. The initial plan for loading needles is stored in the transaction log. Typically, this would be the plan as generated by the dose planning software. An audit trail is made having a record of each and every edit made to the initial plan when the Edit/Add Needles button 232 is used. The loading sequence is recorded to identify which needles (designated by coordinates) were loaded in what order. A completed plan or map of the needles as loaded is optionally recorded. In the event that the automated needle loading system is used to modify needles or load additional needles during an implant procedure, a second audit trail of changes after the needles were originally loaded can also be recorded. If there were changes made during the implant procedure, a completed plan or map of the needles as implanted can also be recorded. Preferably, the Reports button 205 has an option to access the transaction log and create a detailed audit trail of the entire loading process.

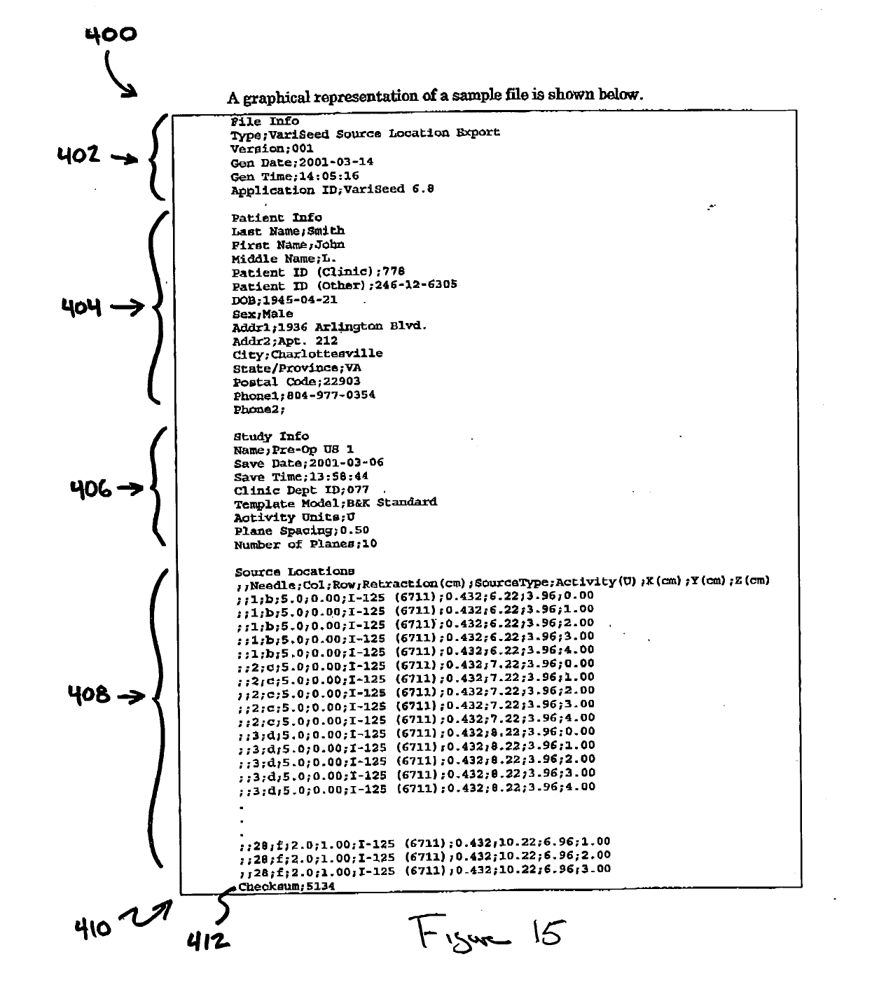

Preferably, the dose planning software outputs a common file transfer format 400 that is accepted by the computer processor 30 for indicating the location the radioisotope seeds are to be loaded. One embodiment of this common file transfer format 400 is shown in FIG. 15. In this embodiment, the file format includes a set of file information 402, a set of patient information 404, a set of study information 406 and a plurality of lines of seed information 408 where each line of information may include multiple variables in comma separated format, with each line being delineated by a semi-colon. The file information 402 provides information about the transfer file 410. The patient information 404 provides identifying and demographic information about the patient. The study information 406 provides information about the volume study on which the dose plan has been based. In this embodiment, the seedplane refers to the retraction plane that identifies the location of each radioisotope seed, e.g., if there are 3 seeds with spacers beginning at 1.0, the seedplane variables would be 1.0, 2.0, 3.0. The locations are rounded off to the nearest 0.5 mm. Preferably, spacers and blanks are automatically inserted by the computer processor 30 and are not represented in the file transfer format 400. Up to ten seedplanes can be specified for a given needle. This same format can be used to record the transaction log, as well as output a common file transfer format to other systems such as an ultrasound system. Preferably, a checksum value 412 is provided at the end of the file format to insure that the information in the transfer format 400 has not been altered or corrupted during transfer.

Another embodiment of the present invention features the ability to automatically convert seedplane information among different needles so that all needles will be loaded from a common zero plane. For example, if ten needles are to be implanted, five of those needles may have a first seed located at seedplane 1.0 and the remaining five needles may have a first seed located at seedplane 2.0. In a preferred embodiment, this process utilizes the common file format 400, although it will be recognized that other representations of how the seeds are stored in the needles could be accommodate by this process. The process determines whether a given location for the zero plane has been established by the user. If not, the file format 400 is examined to find the lowest location of a seedplane of transfer file 410. This location is then set to the zero plane. This process then sets an initial value for the current needle. A processing loop begins by evaluating the beginning seed plane for a current needle. If that beginning seedplane is not equal to the zero plane, then a number of spacers necessary to move the beginning needle to the zero plane are added. A check is made to see whether this is the last needle. If not, the current needle is advanced to the next needle and the loop is continued until all needles have been processed.

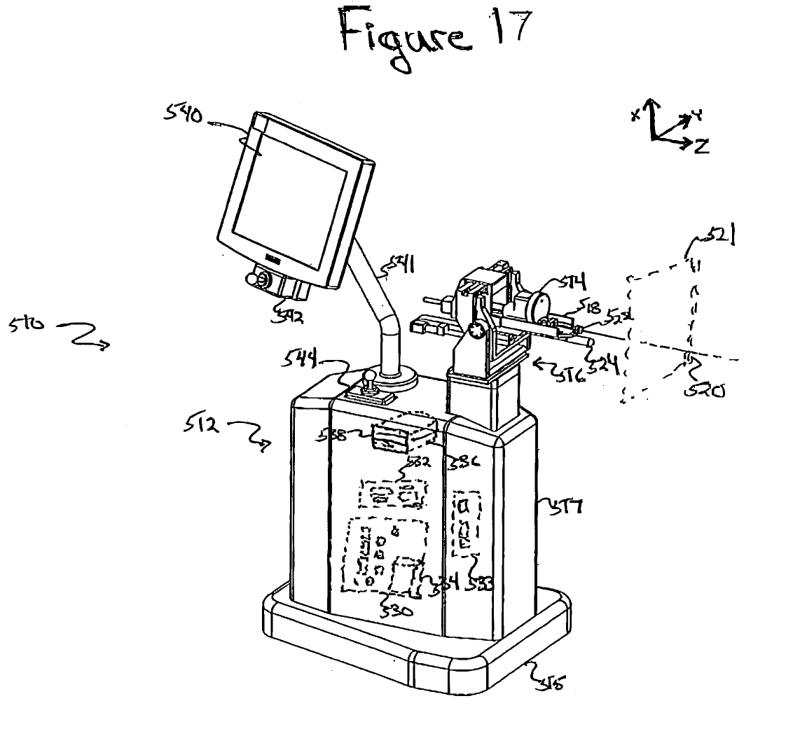

Referring to FIG. 17, an automated implantation system 510 for implanting low dose radioisotope seeds into a patient is comprised of an implantation station 512 into which a replaceable cartridge 514 may be positioned. A moveable assembly 516 is positioned in an appropriate relation to the patient (not shown) for the brachytherapy procedure. A cartridge receiving structure 518 is defined in the moveable assembly 516 along an insertion axis 520. A needle assembly 522 is movable along the insertion axis 520 (in a Z direction) and in a plane 521 defined perpendicular to the insertion axis (in both X and Y directions) by an automated motion control system as will be described. Preferably, an ultrasound probe 524 also carried by the moveable assembly 516 is moveable along an axis parallel to the insertion axis 520.

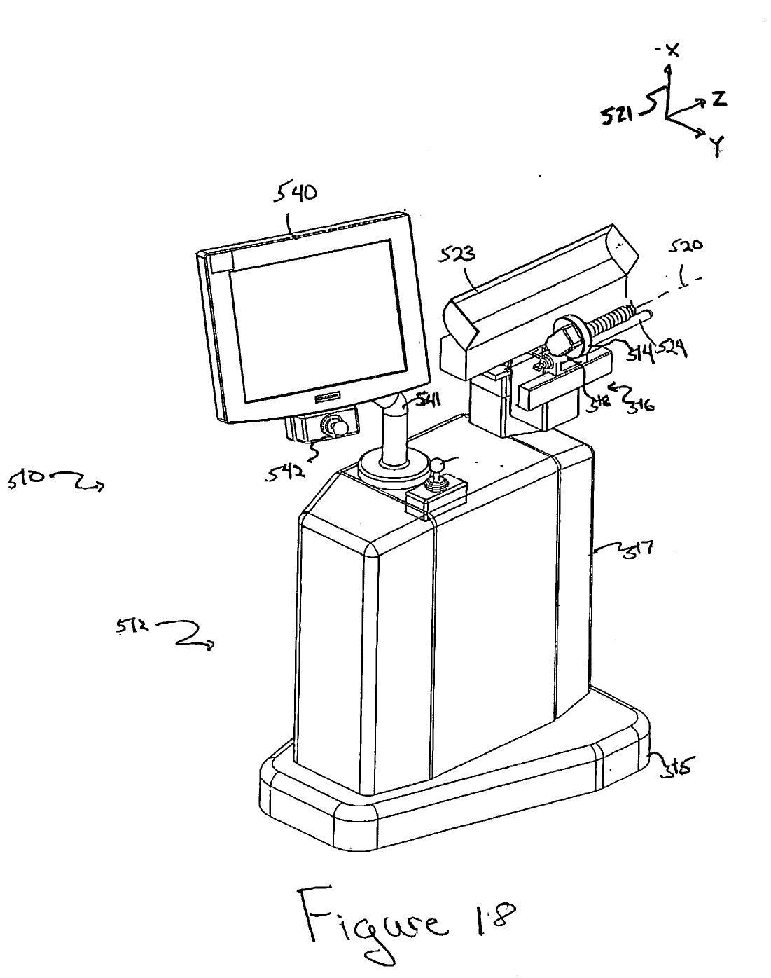

Preferably, the implantation station 512 is a standalone unit that includes a base 515 and a stand 517 supporting the moveable assembly 516 relative to the base 515 (as shown in FIG. 17) and a hinged cover 523 (as shown in FIG. 18) for the moveable assembly 516. All of these components are preferably formed of molded plastic or metal. Although the implantation station 512 will be described as a standalone unit providing its own support and housing arrangements, it will be understood that the automated implantation system 510 of the present invention is equally applicable to an arrangement in which the moveable assembly 516 would be mounted on a table or other platform or where the moveable assembly 516 is hung from an arm or ceiling. Similarly, while the preferred embodiment of the automated implantation system 510 includes all of the electronics, software, controls and displays for operating the implantation station as part of a single unit, the present invention contemplates that the various functions of these components could be performed by separate devices in separate housings.

A computer processor 530 for the automated system is preferably a motherboard having a microprocessor, internal bus, a PCI-compatible bus, DRAM and EPROM or battery backed SRAM, with appropriate external interfaces or mated PC boards for a video interface, multiple channel IDE interfaces, a floppy disk interface, an ethernet interface, COM and LPT interfaces, an external bidirectional parallel port and a serial port. An automated motion control system 532 is preferably a Galil motion controller available from Galil Motion Control Inc. that interfaces to the computer processor 530 via the PCI-compatible bus. The automated motion control system 532 with appropriate software drivers provides all functionality for the lowest level control of stepper motor position and feedback sensors. A hard disc drive 534, floppy disk drive or high density removable media drive 536 and CD or CD-RW drive 538 are also provided for storing data and information to be used by the automated implantation system 510.

A video display 540 that operates as the primary user interface is preferably a 1280 by 1024 resolution flat 18.1 inch flat panel LCD with a resistive touch-screen, such as are available from National Display Systems. In this embodiment, an arm structure 541 positions the display 540 in a position convenient for the user. Alternatively, a conventional non-touch-screen video display and mouse, keyboard or similar input devices could also be provided. Preferably, two separate joy stick controls 542, 544 are provided as direction control input mechanisms to allow a user to control at least the Z-axis direction of the automated motion control system 532. In this embodiment, the joy stick control 542 is preferably a single Z-axis control input located near the video display 540 that controls the advancement and retraction of the needle assembly 522 along the insertion axis 520. The joy stick control 544 is a dual axis control input located on the stand 517 that can selectively control a variety of other automated motion functions for the implantation station 512, including, for example, fine movement of the insertion axis 520 to different location in the X-Y plane 521, as well as gross movements of the moveable assembly relative to the patient. It will be understood that a variety of alternative direction control input mechanisms could also be utilized with the present invention, such as icon controls displayed on the video display 540, voice activated controls processed by the computer processor 530, or switches, slides, dials, or the similar mechanical controls.

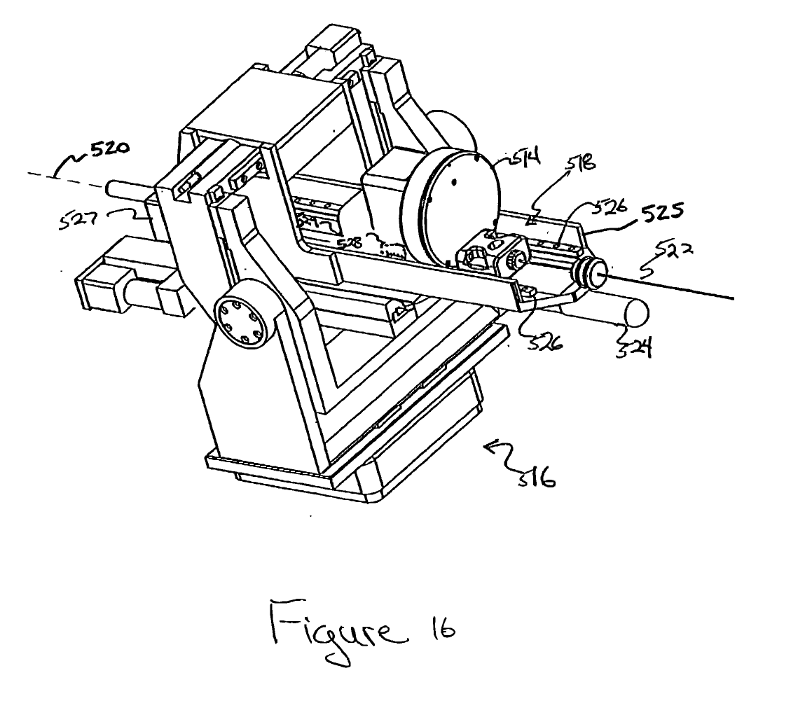

Referring specifically to FIG. 16, the cartridge receiving structure 518 of the preferred embodiment will be described. The cartridge receiving structure 518 includes a U-shaped bracket 525 that rides on a pair of rails 526 with each rail 526 of the bracket preferably being driven by one of a pair of synchronized stepper motors 527. The pair of brackets 525 and corresponding pair of stepper motors 527 are preferably utilized to control any potential skew of the cartridge 514 as it is moved along the insertion axis 520. Alternatively, a single stepper motor and single rail, a linear screw drive, a rodless cylinder or any number of other motion arrangements could be provided to drive the cartridge 514.

Once in position, the implantation station 512 locks the cartridge 514 in place using an electrical solenoid 529 to prevent inadvertent removal of the cartridge 514 during operation of the automated system 510. Locking is initiated automatically once the presence of a cartridge 514 has been detected in the cartridge receiving structure 518 and the user has initiated an implantation operation via display 540. Unlocking the cartridge is initiated by the user selecting a remove cartridge operation via display 540, but only after computer processor 530 has confirmed completion of any critical motions that are part of the implantation operation and removed power to the cartridge 514. Preferably, the only other interface between the cartridge 514 and the cartridge receiving structure 518 is a multiple pin-type electrical connector 528. As the stepper motors and associated encoder discs are contained within the cartridge 514, the need for extremely tight tolerance matches between the cartridge receiving structure 518 and the cartridge 514 is minimized. In addition to the necessary control and sensor signals, the connector 528 includes a ground and power connection to provide power to the cartridge 514. The presence of cartridge 514 in cartridge receiving structure 518 is also detected via a contact on connector 528. Although an arrangement using a bracket 525 and pair of guide rails 526 that is driven by a stepper motor 527 and is connected by the electrical connector 528 and locked by an electrical solenoid 529 is the preferred embodiment for interfacing the cartridge 514 with the cartridge receiving structure 518, it will be recognized that many other structures, such as channels, latches, pivoting arrangements, ball and detent locks, and orientations, such as horizontal or vertical, and connectors, such as optical, infrared, RF, slide contacts, array contacts, or the like, could be used to accomplish the same function of interfacing the cartridge 514 with the cartridge receiving structure 518.

The preferred user interface for the automated implantation station 510 is essentially similar to the user interface as previously described automated loading station 10, except that instead of loading a plurality of implant needles, the buttons are set to implant a plurality of locations. Preferably one screen of the user interface is a system setup and status screen that graphically displays a series of data windows that display current settings of each of the various motion positions for automated implant stations 510. When a user touches one of these data windows, the data window opens to display a control display that allows the user to alter or move the position or state of any axis. Preferably, the Z-axis and X-Y axis are automatically controlled by the computer processor 530 as part of the implantation process as described in the co-pending application.

In one embodiment, the computer processor 530 preferably captures and stores at least one image from the ultrasound probe 524 each time the needle assembly 522 is located at a different position in the plane 521 perpendicular to the insertion axis 520. The computer processor 530 also preferably captures and stores at least one image from the ultrasound probe 524 when the needle assembly 522 is moved forward along the insertion axis 520 to a distal most location where radioisotope seeds will be placed.

In another embodiment, the computer processor 530 includes an autocalibration routine that calibrates an XYZ relationship of the ultrasound probe 524 to the needle assembly 522 each time a different ultrasound probe is used with the automated implantation system 510.

In another embodiment, the computer processor 530 is provided with dose planning software and with image management software that can capture ultrasound images from the ultrasound probe 524. In this embodiment, the motherboard of the computer processor 530 is provided with a frame-grabber daughter board 33 (as shown in FIG. 1B) that interfaces with the ultrasound probe to obtain frame-by-frame image of the prostate gland as the probe is advanced. Preferably, a linear stepper motor is coupled to the probe and to the automated motion control system 532 to allow the image management software to control the movement of the probe. In this way, precise control of the frame-by-frame images used for the volume study can be obtained and the dose plan generated as a result of the volume study can be correlated back to the frame-by-frame images. Preferably, the probe is operated in a similar manner at the time of the brachytherapy procedure and the frame-by-frame images of the volume study can be compared with the current images of the prostate gland. A matching or registration of these two different sets of images can be done manually or with the assistance of the computer processor 530. Once the matching is complete, the dose planning software can compare any changes in the volume or positioning of the prostate gland and update the recommended dose plan accordingly. In this embodiment, as in the preferred embodiment, the number and combination of radioisotope seeds and spacers preloaded into the cartridge 514 can be increased by a given percentage over the minimum number required by the predetermined dose plan to allow for changes to the dose plan as a result of changes to the volume and position of the prostate gland that may occur between the time of the volume study and the time of the brachytherapy procedure. In this embodiment, the physician would utilize the display 540 of the automated system as the display for conducting the volume study and monitoring the brachytherapy procedure, as well as for controlling the automatic loading of the implant needles.

It should be understood that in the broadest sense, the automated motion control system of the present invention encompasses the various motors, actuators, encoders, detectors, and feedback circuits that accomplish the controlled motion required to load the implant needles automatically and without manual intervention. It will be recognized by a person of ordinary skill in the art that numerous variations in the arrangement of motors, actuators, encoders, detectors, and feedback circuits can be made and still accomplish the function of loading the implant needles automatically, such as belt driven systems or screw-drive powered systems instead of direct motor driven systems, mechanical or electrical encoders, and detectors instead of optical encoders and detectors, and linear actuators instead of rotary actuators or vice versa.

Although the preferred embodiment of the automated system of the present invention has been described, it will be recognized that numerous changes and variations can be made and that the scope of the present invention is intended to be defined by the claims.

1. An automated system for ejecting radioisotope seeds into an implant needle comprising:
at least one source of a plurality of radioisotope seeds to be selectively ejected from at least one aperture; and
a station operably connected to the at least one source and including a computer processor having a user interface that displays information about the automated system and accepts commands from a user to control the process of ejecting the radioisotope seeds into the implant needle by controlling an automated motion control system that selectively ejects radioisotope seeds from the at least one aperture into the implant needle,
wherein the user interacts with the user interface to enter commands to alter a predetermined dose plan such that the computer processor dynamically determines the radioisotope seeds to be selectively ejected into the implant needle in response to the predetermined dose plan and the commands.

2. The automated system of claim 1 wherein the source of the plurality of radioisotope seeds is a replaceable cartridge having a machine readable storage medium that stores indicia representing at least the quantity and location of the plurality of radioisotope seeds preloaded into the cartridge and wherein the computer processor controls the automated motion control system to selectively eject radioisotope seeds from the cartridge in response to the indicia.

3. The automated system of claim 1 wherein the user interface is a touch-screen display.

4. The automated system of claim 1 wherein a plurality of implant needles are to be loaded and wherein the user interface displays a graphic representation of the coordinates of each needle to be loaded in accordance with the predetermined dose plan and a user selects the next needle to be loaded by indicating one of the coordinates.

5. The automated system of claim 4 wherein each coordinate has an icon associated therewith and as a coordinate is selected a graphical characteristic of the icon is changed.

6. The automated system of claim 1 wherein a single implant needles is to be implanted and wherein the user interface displays a graphic representation of the coordinates of each of a plurality of locations to be implanted in accordance with the predetermined dose plan and a user selects the next location to be implanted by indicating one of the coordinates.

7. The automated system of claim 6 wherein each coordinate has an icon associated therewith and as a coordinate is selected a graphical characteristic of the icon is changed.

8. The automated system of claim 1 wherein the user interface displays a graphic representation of a cross-section of the implant needle as the radioisotope seeds dispensed from the source to allow a user to confirm visually the proper loading of radioisotope seeds within the implant needle.

9. A method for ejecting low dose radioisotope seeds into an implant needle comprising:
(a) providing at least one source of a plurality of radioisotope seeds to be selectively ejected from at least one aperture; and
(b) providing a computer processor having a user interface that:
(b1) displays information about the radioisotope seeds, including information from a predetermined dose plan; and
(b2) accepts commands from a user, including commands to selectively modify the predetermined dose plan;
(c) using the computer processor to control loading the radioisotope seeds into the implant needle by controlling an automated motion control system that selectively ejects radioisotope seeds from the at least one aperture into the implant needle in accordance with the predetermined dose plan as modified by the commands from the user.

10. The method of claim 9 wherein the source of the plurality of radioisotope seeds in step (a) is a replaceable cartridge having a machine readable storage medium that stores indicia representing at least the quantity and location of the plurality of radioisotope seeds preloaded into the cartridge and wherein the computer processor controls the automated motion control system in step (c) to selectively eject radioisotope seeds from the cartridge in response to the indicia.

11. The method of claim 9 wherein a plurality of implant needles are to be loaded and wherein the user interface in step (b1) displays a graphic representation of a coordinate of each needle to be loaded in accordance with a predetermined dose plan and in step (b2) a user selects the next needle to be loaded by indicating one of the coordinates.

12. The method of claim 11 wherein each coordinate has an icon associated therewith that is displayed in step (b1) and as a coordinate is selected in step (b2) a graphical characteristic of the icon is changed on the graphic representation.

13. The method of claim 9 wherein a single implant needles is to be loaded and wherein the user interface in step (b1) displays a graphic representation of a coordinate of each of a plurality of locations to be implanted in accordance with the predetermined dose plan and in step (b2) a user selects the next location to be loaded by indicating one of the coordinates.

14. The method of claim 13 wherein each coordinate has an icon associated therewith that is displayed in step (b1) and as a coordinate is selected in step (b2) a graphical characteristic of the icon is changed on the graphic representation.

15. The method of claim 9 wherein the user interface displays a graphic representation in step (b1) of a cross-section of the implant needle as the radioisotope seeds dispensed from the source in step (c) to allow a user to confirm visually the proper loading of radioisotope seeds within the implant needle.

16. An automated system for ejecting radioisotope seeds into an implant needle comprising:
means for storing a plurality of radioisotope seeds, including means for selectively automatically ejecting at least one radioisotope seed from at least one aperture; and
computer processing means for displaying information about the automated system and for accepting commands from a user to control the means for selectively ejecting the radioisotope seeds by altering a predetermined dose plan communicated to the computer processing means.

17. An automated system for ejecting low dose radioisotope seeds into an implant needle comprising:
at least one source of a plurality of radioisotope seeds;
a station operably connected to the at least one source and including a computer processor having a user interface that displays information about the automated system and accepts commands from a user to control the process of ejecting the radioisotope seeds into the implant needle by displaying a graphic representation of the coordinates of each of a plurality of locations in accordance with a predetermined dose plan using an icon and having a user select a location by indicating one of the coordinates on the graphic representation and as the coordinate is selected changing a graphical characteristic of the icon associated with that coordinate.

18. The automated system of claim 17 wherein the station is selected from the set consisting of a loading station for loading radioisotopes seeds into a plurality of implant needles to be implanted by a manual procedure and an implantation station for ejecting radioisotope seeds into a single implant needle to be implanted by an automated procedure.

19. A method for ejecting low dose radioisotope seeds into an implant needle, the method comprising:
providing at least one source of a plurality of radioisotope seeds;
providing a station operably connected to the at least one source and including a computer processor having a user interface that displays information about the automated system and accepts commands from a user to control the process of ejecting the radioisotope seeds into the implant needle;
displaying on the user interface a graphic representation of coordinates of each of a plurality of locations in accordance with a predetermined dose plan using an icon;
having a user select a location by indicating one of the coordinates on the graphic representation;
changing a graphical characteristic of the icon associated with the coordinate as the coordinate is selected.

20. An automated system for ejecting low dose radioisotope seeds into an implant needle comprising:
means for storing a plurality of radioisotope seeds;
computer processing means operably connected to the means for storing for displaying information about the automated system and the radioisotope, including means for displaying a graphic representation of coordinates of each of a plurality of locations in accordance with a predetermined dose plan using an icon and, in response to a user selecting one of the locations by indicating one of the coordinates on the graphic representation, changing a graphical characteristic of the icon associated with that coordinate.

21. An automated system for ejecting low dose radioisotope seeds into an implant needle comprising:
at least one source of a plurality of radioisotope seeds;
a station operably connected to the at least one source;
a computer processor operably connected to the station to control the operation of the station; and
a touch-screen user interface operably connected to the computer processor that displays information about the automated system and accepts commands from a user to control the process of loading the radioisotope seeds into the implant needle.

22. The automated system of claim 21 wherein the touch-screen user interface displays a graphic representation of the coordinates of a plurality of locations in accordance with a predetermined dose plan using an icon and a user selects a location by indicating one of the coordinates on the graphic representation and as the coordinate is selected a graphical characteristic of the icon associated with that coordinate is changed.

23. The automated system of claim 21 wherein the station is selected from the set consisting of a loading station for loading radioisotopes seeds into a plurality of implant needles to be implanted by a manual procedure and an implantation station for ejecting radioisotope seeds into a single implant needle to be implanted by an automated procedure.

24. A method for ejecting low dose radioisotope seeds into an implant needle, the method comprising:
providing at least one source of a plurality of radioisotope seeds;
providing a station operably connected to the at least one source;
providing a computer processor operably connected to the station for controlling the operation of the station;
providing a touch-screen user interface operably connected to the computer processor that displays information about the automated system and accepts commands from a user to control the process of loading the radioisotope seeds into the implant needle.

25. An automated system for loading low dose radioisotope seeds into a plurality of implant needles comprising:
at least one source of a plurality of radioisotope seeds;
a loading station operably connected to the at least one source;
a computer processor operably connected to the loading station to control the operation of the loading station;
a user interface operably connected to the computer processor that displays information about the automated system and accepts commands from a user to control the process of loading the radioisotope seeds into the implant needles; and
a software routine executing on the computer processor to perform a transformation of a predetermined dose plan having implant needles loaded at different starting retraction planes to a modified dose plan in which all implant needles have a common zero retraction plane by examining the dose plan for each implant needle and 14 inserting additional spacers in all implant needles having a starting retraction plane that is not equal to the common zero retraction plane.

26. The automated system of claim 25 wherein the software routine examines the predetermined dose plan to determine a value to be used as the common zero retraction plane.

27. The automated system of claim 25 wherein the station is selected from the set consisting of a loading station for loading radioisotopes seeds into a plurality of implant needles to be implanted by a manual procedure and an implantation station for ejecting radioisotope seeds into a single implant needle to be implanted by an automated procedure.

28. A method for loading low dose radioisotope seeds into an implant needle using an automated loading station having at least one source of a plurality of radioisotope seeds and a computer processor having a user interface for controlling the automated loading station, the method comprising:
(a) providing a predetermined dose plan to the computer processor having indications to load implant needles at different starting retraction planes; and
(b) using the computer processor to transform the predetermined dose plan into a modified dose plan in which all implant needles have a common zero retraction plane by examining the dose plan for each implant needle and inserting additional spacers in all implant needles having a starting retraction plane that is not equal to the common zero retraction plane.

29. The method of claim 28 wherein step (b) further includes the step of examining the predetermined dose plan to determine a value to be used as the common zero retraction plane.

30. An automated system for loading low dose radioisotope seeds into an implant needle in response to a predetermined dose plan to having indications to load implant needles at different starting retraction planes, the automated system comprising:
means for selectively automatically loading of a plurality of radioisotope seeds into a plurality of implant needles; and
computer processing means operably connected to the means for loading for transforming the predetermined dose plan into a modified dose plan in which all implant needles have a common zero retraction plane by examining the dose plan for each implant needle and inserting additional spacers in all implant needles having a starting retraction plane that is not equal to the common zero retraction plane.

31. An automated system for implanting low dose radioisotope seeds at a plurality of locations using an implant needle comprising:
at least one source of a plurality of radioisotope seeds;
an implantation station operably connected to the at least one source;
a computer processor operably connected to the implantation station to control the operation of the implantation station;
a user interface operably connected to the computer processor that displays information about the automated system and accepts commands from a user to control the process of implanting the radioisotope seeds at the plurality of locations using the implant needle; and
a software routine executing on the computer processor to perform a transformation of a predetermined dose plan having locations at different starting retraction planes to a modified dose plan in which all locations have a common zero retraction plane by examining the dose plan for each location and inserting additional spacers in the implant needle for those locations having a starting retraction plane that is not equal to the common zero retraction plane.

32. The automated system of claim 31 wherein the software routine examines the predetermined dose plan to determine a value to be used as the common zero retraction plane.

33. A method for implanting low dose radioisotope seeds at a plurality of locations using an implant needle controlled by an automated implantation station having at least one source of a plurality of radioisotope seeds and a computer processor having a user interface for controlling the automated implantation station, the method comprising:
(a) providing a predetermined dose plan to the computer processor having indications to implant radioisotope seeds at the plurality of locations where at least two locations have different starting retraction planes; and
(b) using the computer processor to transform the predetermined dose plan into a modified dose plan in which all the locations have a common zero retraction plane.

34. The method of claim 33 wherein step (b) is performed by examining the dose plan for each of the plurality of locations and inserting additional spacers in the implant needle for each location having a starting retraction plane that is not equal to the common zero retraction plane.

35. An automated system for implanting low dose radioisotope seeds at a plurality of locations using an implant needle comprising:
at least one source of a plurality of radioisotope seeds;
an implantation station operably connected to the at least one source and including an ultrasound probe;
a computer processor operably connected to the implantation station to control the operation of the implantation station;
a user interface operably connected to the computer processor that displays information about the automated system and accepts commands from a user to control the process of implanting the radioisotope seeds at the plurality of locations using the implant needle; and
a software routine executing on the computer processor that monitors information from the ultrasound probe and, in response, selectively adjusts a location of a base retraction plane from which each of the plurality of locations are to be implanted.

36. The automated system of claim 35 wherein the station is selected from the set consisting of a loading station for loading radioisotopes seeds into a plurality of implant needles to be implanted by a manual procedure and an implantation station for ejecting radioisotope seeds into a single implant needle to be implanted by an automated procedure.

37. A method for implanting low dose radioisotope seeds at a plurality of locations using an implant needle, the method comprising:
providing at least one source of a plurality of radioisotope seeds;
providing an implantation station operably connected to the at least one source and including an ultrasound probe;
providing a computer processor operably connected to the implantation station for controlling the operation of the implantation station;
providing a user interface operably connected to the computer processor;
displaying information about the automated system on the user interface;
accepting commands via the user interface from a user to control the process of implanting the radioisotope seeds at the plurality of locations using the implant needle;
providing a software routine executing on the computer processor; and
using the software routine for monitoring information from the ultrasound probe; and
selectively adjusting a location of a base retraction plane from which each of the plurality of locations are to be implanted in response to the monitored information.

38. An automated system for implanting low dose radioisotope seeds at a plurality of locations in a patient comprising:
means for generating an ultrasound information associated with the plurality of locations;
means for automatically selectively implanting a plurality of radioisotope seeds into the plurality of locations, including means for monitoring the ultrasound information and, in response, selectively adjusting a location of a base retraction plane from which each of the plurality of locations are to be implanted.

39. An automated system for loading a plurality of low dose radioisotope seeds for implantation into a patient comprising:
at least one source of a plurality of radioisotope seeds;
an implantation station operably connected to the at least one source;
a computer processor operably connected to the implantation station to control the operation of the implantation station in response to a predetermined dose plan;
a user interface operably connected to the computer processor that displays information about the automated system and accepts commands from a user to control the computer processor and make alterations to the dose plan; and
a software routine executing on the computer processor that creates a transaction log documenting all of the alternations to the dose plan by the user.

40. The automated system of claim 39 wherein the station is selected from the set consisting of a loading station for loading radioisotopes seeds into a plurality of implant needles to be implanted by a manual procedure and an implantation station for ejecting radioisotope seeds into a single implant needle to be implanted by an automated procedure.

41. A method for implanting low dose radioisotope seeds at a plurality of locations using an implant needle, the method comprising:
providing at least one source of a plurality of radioisotope seeds;
providing a computer processor operably connected to the implantation station for controlling the operation of ejecting radioisotope seeds from the at least one source;
providing a user interface operably connected to the computer processor;
displaying information about the automated system on the user interface;
providing a predetermined dose plan according to which the radioisotope seeds are to be implanted
accepting commands via the user interface from a user to control the computer processor and make alterations to the dose plan;
providing a software routine executing on the computer processor that creates a transaction log documenting all of the alternations to the dose plan by the user.

42. An automated system for implanting low dose radioisotope seeds at a plurality of locations in a patient comprising:
means for ejecting a plurality of radioisotope seeds into at least one implant needle;
computer processing means for controlling the means for ejecting in response to commands from a user to modify a predetermined dose plan for implanting the radioisotope seeds, including means for creating a transaction log documenting all of the modification to the dose plan by the user.
